(19) United States
(12) Patent Application Publication
(10) Pub. No.: US 2023/0151108 A1
(43) Pub. Date: May 18, 2023

(54) BISPECIFIC ANTIBODIES AGAINST CD9 AND CD137

(71) Applicant: UCB BIOPHARMA SRL, Brussels (BE)

(72) Inventors: David Alan COOK, Slough, Berkshire (GB); Helen Margaret FINNEY, Slough, Berkshire (GB); Stephen Edward RAPECKI, Slough, Berkshire (GB)

(21) Appl. No.: 17/929,026

(22) PCT Filed: Feb. 13, 2020
(86) PCT No.: PCT/EP2020/053771

§ 371 Date: Aug. 10, 2022

(51) Int. Cl. C07K 16/28; C07K 14/765; A61P 35/00

(52) U.S. Cl. CPC C07K 16/2896; C07K 16/2878; C07K 14/765; A61P 35/00; A61K 2039/505

(57) ABSTRACT

The present invention relates to multispecific antibodies against a novel targets' combination of CD137 and CD9, and their use in the treatment of cancer and infectious diseases.

### FIELD OF THE INVENTION

The present invention belongs to the field of multispecific antibodies binding at least CD137 and CD9, and their uses in the treatment of cancer and/or infectious diseases.

### BACKGROUND OF THE INVENTION

T cells are key to a successful cell-mediated immune response necessary to eliminate cancer cells, bacteria and viruses. They recognise antigens displayed on the surface of tumour cells or antigens from bacteria and viruses replicating within the cells or from pathogens or pathogen products endocytosed from the extracellular fluid. T cells have two major roles. They can become cytotoxic T cells capable of destroying cells marked as foreign. Cytotoxic T cells have a unique surface protein called CD8, thus they are often referred to as CD8+ T cells. Alternatively, T cells can become helper T cells, which work to regulate and coordinate the immune system. Helper T cells have a unique surface protein called CD4 and are thus often called CD4+ T cells. Helper T cells have several important roles in the immune system: 1) responding to activation by specific antigens by rapidly proliferating; 2) signaling B cells to produce antibodies; and 3) activating macrophages.

Cancer eludes the immune system by exploiting mechanisms developed to avoid auto-immunity. However, the immune system is programmed to avoid immune over-activation which could harm healthy tissue. T-cell activation is at the core of these mechanisms. Antigen specific T cells normally able to fight disease can become functionally tolerant (exhausted) to infectious agents or tumour cells by over stimulation or exposure to suppressive molecules. Therefore, molecules that enhance the natural function of T cells or overcome suppression of T cells have great utility in the treatment or prevention of cancer and infectious disease.

In recent years, immunotherapy has become an established treatment option for an increasing number of cancer patients, exemplified by the increased use of therapeutic antibody-based immune checkpoint inhibitors (CPI's). This has arisen from an increased immunological understanding of how cancer cells perturb immune cell activation by hijacking pathways normally involved in maintaining tolerance and skewing the balance between co-stimulation and co-inhibition (Chen and Mellman., Immunity. 39:1-105 (2013)). Amongst the pathways that have emerged as key regulators in this regard, include CTLA-4 and the PD-1/PD-L1 checkpoint molecules serving to down-regulate T cell and myeloid cell activation in the tumour microenvironment. Ipilimumab (anti-CTLA-4) was the first CPI to be approved in 2011 as a treatment for melanoma, closely followed by FDA approval of anti-PD1 directed antibodies, pembrolizumab and nivolumab in 2014 (Hargadon et al., International Immunopharmacol. 62:29-39 (2018)). There are still significant challenges in understanding differences in efficacy across patient groups, ranging from complete responses, to treatment relapse and even failure to respond, (Haslam and Prasad. JAMA Network Open.5:2e192535 (2019)). Despite the promising anti-tumour efficacy of several monoclonal antibodies, the successful treatment of many cancers is limited in treatments with a single antibody. For example, clinical trials of two agonist antibodies utomilumab (PF-05082566) and urelumab (BMS-663513), both targeting CD137 (4-1BB, TNFRSF9), an inducible costimulatory receptor expressed on activated T cells, are ongoing in multiple cancer indications. Clinical success for anti-CD137 antibody monotherapy may prove a complex balance between efficacy and toxicity. Urelumab has demonstrated dose-liming toxicity in phase I and II clinical trials (Segal, N. H. et al Clin. Cancer Res. 23, 1929-1936 (2017)) which utomilumab has not in phase I (Tolcher, A. W. et al. Clin. Cancer Res. 23, 5349-5357 (2017); Segal, N. H. et al. J. Clin. Oncol. 32 (suppl.) 3007 (2017): Gopal, A. K. et al. J. Clin. Oncol. 33 (suppl) 3004 (2017).

Combinations of two or more antibodies are currently being tested in patients to provide improved methods of treatment. To date, these therapies rely on rational design of known mechanisms of action and are largely based on combining antigen-specificities known to be independently effective in the treatment of cancer, either as combination therapies or in a bispecific antibody format. This state of the art approach is a limiting factor in the development of new therapies as it relies on known therapies.

Therefore, there is still the need to identify novel modulators of T-cell activation based on unbiased biology, which allows identification of novel target pairs capable of enhancing T-cell activation and induction of T-cell proliferation for the treatment of cancer and infectious diseases.

### SUMMARY OF THE INVENTION

The present invention addresses the above-identified need by providing in a first aspect an antibody which comprises a first antigen-binding portion binding CD137 and a second antigen-binding portion binding CD9.

In one embodiment of this first aspect, each of the antigen-binding portions of the antibody which comprises a first antigen-binding portion binding CD137 and a second antigen-binding portion binding CD9 is a monoclonal antigen-binding portion.

In another embodiment, each of the antigen-binding portions is independently selected from a Fab, a Fab′, a scFv or a VHH. In yet another aspect, the antigen-binding portions are the antigen-binding portions of an IgG.

In another embodiment, the antibody which comprises a first antigen-binding portion binding CD137 and a second antigen-binding portion binding CD9, is chimeric, human or humanised, and preferably the antibody is humanised.

In another embodiment, the antibody comprises a heavy chain constant region selected from an IgG1, an IgG2, IgG3 or an IgG4 isotype, or a variant thereof.

In another embodiment, the antibody further comprises at least an additional antigen-binding portion. The additional antigen-binding portion may be capable of increasing the half-life of the antibody. Preferably the additional antigen-binding portion binds albumin, more preferably human serum albumin.

In one embodiment, the first antigen binding portion binds CD137 in CD137 domain CRD1, wherein preferably the first antigen-binding portion binds within amino acids 25 to 68 of SEQ ID NO: 1, more preferably within amino acids 25 to 45 of SEQ ID NO: 1 and/or the second antigen binding portion binds CD9 in CD9 loop 2, wherein preferably the second antigen-binding portion binds within amino acids 112 to 195 of SEQ ID NO: 2.

In one embodiment, the first antigen-binding portion binding CD137 of the antibody of the present invention comprises a first heavy chain variable region and a first light chain variable region and the second antigen-binding portion binding CD9 comprises a second heavy chain variable region and a second light chain variable region and wherein:
a. The first heavy chain variable region comprises a CDR-H1 comprising SEQ ID NO: 3, a CDR-H2 comprising SEQ ID NO: 4 and a CDR-H3 comprising SEQ ID NO: 5; and
b. The first light chain variable region comprises a CDR-L1 comprising SEQ ID NO: 6, a CDR-L2 comprising SEQ ID NO: 7 and a CDR-L3 comprising SEQ ID NO: 8; and
c. The second heavy chain variable region comprises a CDR-H1 comprising SEQ ID NO: 9, a CDR-H2 comprising SEQ ID NO: 10 and a CDR-H3 comprising SEQ ID NO: 11; and
d. The second light chain variable region comprises a CDR-L1 comprising SEQ ID NO: 12, a CDR-L2 comprising SEQ ID NO: 13 and a CDR-L3 comprising SEQ ID NO: 14;
or
e. The first heavy chain variable region comprises SEQ ID NO: 15 and the first light chain variable region comprises SEQ ID NO: 17; and the second heavy chain variable region comprises SEQ ID NO: 19 and second light chain variable region comprises SEQ ID NO: 21;
or
f. The first heavy chain variable region is encoded by a nucleotide sequence comprising SEQ ID NO: 16 and the first light chain variable region is encoded by a nucleotide sequence comprising SEQ ID NO: 18; and the second heavy chain variable region is encoded by a nucleotide sequence comprising SEQ ID NO: 20 and second light chain variable region is encoded by a nucleotide sequence comprising SEQ ID NO: 22.

In a second aspect of the present invention there is provided a pharmaceutical composition comprising the antibody according to the first aspect of the invention and all its embodiments and one or more pharmaceutically acceptable excipients.

In a third aspect, the invention provides for the antibody according to the first aspect of the invention and all its embodiments or the pharmaceutical composition according to the second aspect of the invention and all its embodiments for use in therapy.

In one embodiment of this third aspect, the use is for the treatment of cancer and/or an infectious disease. In another embodiment, the antibody or the composition according to the invention and all its embodiments are for use in the treatment of cancer concomitantly or sequentially to one or more additional cancer therapies.

In a fourth aspect of the present invention, there is provided for, a method for treating a subject afflicted with cancer and/or an infectious disease, comprising administering to the subject a pharmaceutically effective amount of the antibody according to the first aspect of the invention and all its embodiments or the pharmaceutical composition according to the second aspect of the invention and all its embodiments.

In one embodiment of this fourth aspect, the antibody or the composition are administered concomitantly or sequentially to one or more additional cancer therapies.

In a fifth aspect, the invention provides for the use of an antibody according to the first aspect of the invention and all its embodiments or the pharmaceutical composition according to the second aspect of the invention and all its embodiments in the manufacture of a medicament for treating cancer.

In one embodiment of this fifth aspect, the antibody or the composition are administered concomitantly or sequentially to one or more additional cancer therapies.

### BRIEF DESCRIPTION OF THE DRAWINGS

FIG. 1. General representation of a Fab-X and Fab-Y comprising antigen-binding portions and of the resulting bispecific antibody.

FIG. 2. Log 2 fold change in the median fluorescence intensity (MFI) of CD25 expression on CD8+ T-cells in the presence of anti-CD3 (10 ng/mL) stimulation. Samples of the stimulated cultured peripheral blood mononuclear cell (PBMC) were analysed upon treatment with CD137-CD9 bispecific antibodies or control antibodies by flow cytometry and the CD8+ T-cell population identified. Log 2 fold changes were calculated for the MFI of CD25 levels in the treated samples relative to the anti-CD3 stimulated controls. N=4 donors, 2 technical replicates±standard error of the mean (SEM).

FIG. 3. Log 2 fold change in the MFI of CD25 expression on CD8+ T cells in the absence of any stimulation. Samples of the cultured PBMC were analysed upon treatment with CD137-CD9 bispecific antibodies or control antibodies by flow cytometry and the CD8+ T-cell population identified. Log 2 fold changes were calculated for the MFI of CD25 levels in the treated samples relative to the controls. N=4 donors, 2 technical replicates±SEM.

FIG. 4. Log 2 fold change in the MFI of CD71 expression on CD8+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD8+ T-cell population identified. Log 2 fold changes were calculated for the MFI of CD71 levels in the treated samples relative to the anti-CD3 stimulated controls. N=4 donors, 2 technical replicates±SEM.

FIG. 5. Log 2 fold change in the MFI of CD71 expression on CD8+ T cells in the absence of any stimulation. Samples of the cultured PBMC were analysed upon treatment with CD137-CD9 bispecific antibodies or control antibodies by flow cytometry and the CD8+ T-cell population identified. Log 2 fold changes were calculated for the MFI of CD71 levels in the treated samples relative to the controls. N=4 donors, 2 technical replicates±SEM.

FIG. 6. Log 2 fold change in the MFI of CD137 expression on CD8+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD8+ T-cell population identified. Log 2 fold changes were calculated for the MFI of CD137 levels in the treated samples relative to the anti-CD3 stimulated controls. N=4 donors, 2 technical replicates±SEM.

FIG. 7. Log 2 fold change in the MFI of CD137 expression on CD8+ T cells in the absence of any stimulation. Samples of the cultured PBMC were analysed upon treatment with CD137-CD9 bispecific antibodies or control antibodies by flow cytometry and the CD8+ T-cell population identified. Log 2 fold changes were calculated for the MFI of CD137 levels in the treated samples relative to the controls. N=4 donors, 2 technical replicates±SEM.

FIG. 8. Log 2 fold change in the MFI of CD25 expression on CD4+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD4+ T-cell population identified. Log 2 fold changes were calculated for the MFI of CD25 levels in the treated samples relative to the anti-CD3 stimulated controls. N=4 donors, 2 technical replicates±SEM.

FIG. 9. Log 2 fold change in the MFI of CD25 expression on CD4+ T cells in the absence of any stimulation. Samples of the cultured PBMC were analysed upon treatment with CD137-CD9 bispecific antibodies or control antibodies by flow cytometry and the CD4+ T-cell population identified. Log 2 fold changes were calculated for the MFI of CD25 levels in the treated samples relative to the controls. N=4 donors, 2 technical replicates±SEM.

FIG. 10. Log 2 fold change in the MFI of CD71 expression on CD4+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD4+ T-cell population identified. Log 2 fold changes were calculated for the MFI of CD71 levels in the treated samples relative to the anti-CD3 stimulated controls. N=4 donors, 2 technical replicates±SEM.

FIG. 11. Log 2 fold change in the MFI of CD71 expression on CD4+ T cells in the absence of any stimulation. Samples of the cultured PBMC were analysed upon treatment with CD137-CD9 bispecific antibodies or control antibodies by flow cytometry and the CD4+ T-cell population identified. Log 2 fold changes were calculated for the MFI of CD71 levels in the treated samples relative to the controls. N=4 donors, 2 technical replicates±SEM.

FIG. 12. Log 2 fold change in the MFI of CD137 expression on CD4+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD4+ T-cell population identified. Log 2 fold changes were calculated for the MFI of CD137 levels in the treated samples relative to the anti-CD3 stimulated controls. N=4 donors, 2 technical replicates±SEM.

FIG. 13. Log 2 fold change in the MFI of CD137 expression on CD4+ T cells in the absence of any stimulation. Samples of the cultured PBMC were analysed upon treatment with CD137-CD9 bispecific antibodies or control antibodies by flow cytometry and the CD4+ T-cell population identified. Log 2 fold changes were calculated for the MFI of CD137 levels in the treated samples relative to the controls. N=4 donors, 2 technical replicates±SEM.

FIG. 14. Log 2 fold change in the MFI of CD25 expression on CD8+ T cells in the presence of anti-CD3 (10 ng/mL) stimulation. PBMC cultures were treated with soluble anti-CD3 (10 ng/mL) for 48 hours in the presence of either the CD137-CD9 bispecific construct, control constructs or corresponding IgG proteins. The samples were then analysed by flow cytometry and the CD8+ T cell population identified. Log 2 fold changes were calculated for the MFI of CD25 levels in the treated samples relative to the anti-CD3 stimulated controls. N=4 donors, 2 technical replicates±SEM.

FIG. 15. Log 2 fold change in the MFI of CD25 expression on CD8+ T cells in the absence of any stimulation. PBMC cultures were incubated for 48 hours in the presence of either the CD137-CD9 bispecific antibodies, control antibodies or corresponding IgG proteins. The samples were then analysed by flow cytometry and the CD8+ T cell population identified. Log 2 fold changes were calculated for the MFI of CD25 levels in the treated samples relative to the untreated controls. N=4 donors, 2 technical replicates±SEM.

FIG. 16. Log 2 fold change in the MFI of CD25 expression on CD4+ T cells in the presence of anti-CD3 (10 ng/mL) stimulation. PBMC cultures were treated with soluble anti-CD3 (10 ng/mL) for 48 hours in the presence of either the CD137-CD9 bispecific antibodies, control antibodies or corresponding IgG proteins. The samples were then analysed by flow cytometry and the CD4+ T cell population identified. Log 2 fold changes were calculated for the MFI of CD25 levels in the treated samples relative to the anti-CD3 stimulated controls. N=4 donors, 2 technical replicates±SEM.

FIG. 17. Log 2 fold change in the MFI of CD25 expression on CD4+ T cells in the absence of any stimulation. PBMC cultures were incubated for 48 hours in the presence of either the CD137-CD9 bispecific antibodies, control antibodies or corresponding IgG proteins. The samples were then analysed by flow cytometry and the CD4+ T cell population identified. Log 2 fold changes were calculated for the MFI of CD25 levels in the treated samples relative to the untreated controls. N=4 donors, 2 technical replicates±SEM.

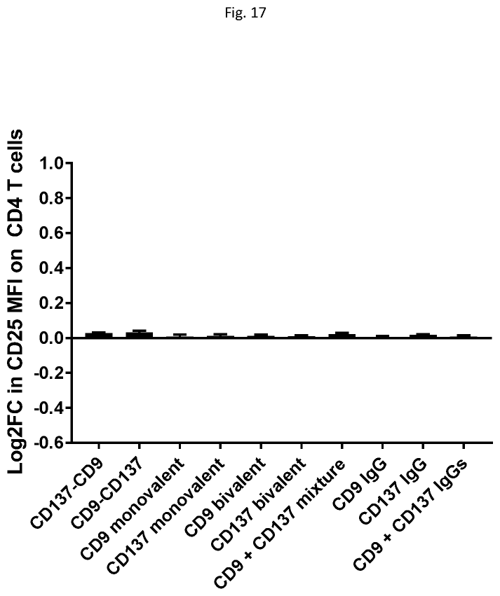

FIG. 18. Log 2 fold change in the MFI of CD71 expression on CD8 T cells in the presence of anti-CD3 (10 ng/mL) stimulation. PBMC cultures were treated with soluble anti-CD3 (10 ng/mL) for 48 hours in the presence of either the CD137-CD9 bispecific antibodies, control antibodies or corresponding IgG proteins. The samples were then analysed by flow cytometry and the CD8 T cell population identified. Log 2 fold changes were calculated for the MFI of CD71 levels in the treated samples relative to the anti-CD3 stimulated controls. N=4 donors, 2 technical replicates±SEM.

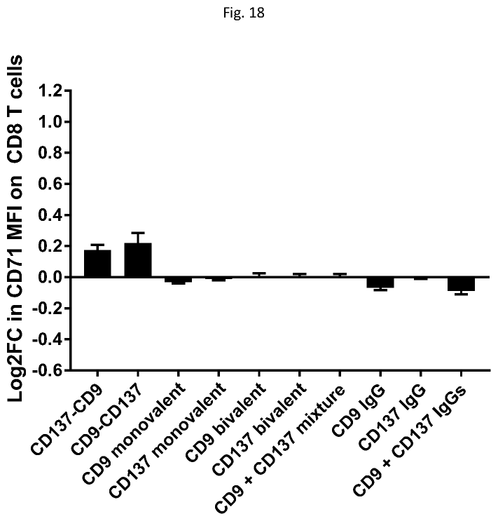

FIG. 19. Log 2 fold change in the MFI of CD71 expression on CD8 T cells in the absence of any stimulation. PBMC cultures were incubated for 48 hours in the presence of either the CD137-CD9 bispecific antibodies, control antibodies or corresponding IgG proteins. The samples were then analysed by flow cytometry and the CD8 T cell population identified. Log 2 fold changes were calculated for the MFI of CD71 levels in the treated samples relative to the untreated controls. N=4 donors, 2 technical replicates±SEM.

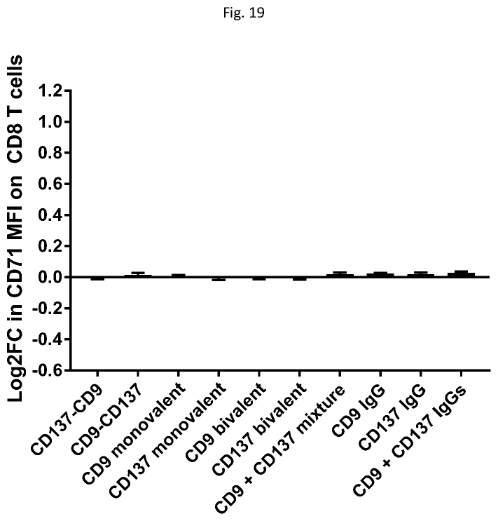

FIG. 20. Log 2 fold change in the MFI of CD71 expression on CD4 T cells in the presence of anti-CD3 (10 ng/mL) stimulation. PBMC cultures were treated with soluble anti-CD3 (clone UCHT-1) at 10 ng/mL for 48 hours in the presence of either the CD137-CD9 bispecific antibodies, control antibodies or corresponding IgG proteins. The samples were then analysed by flow cytometry and the CD4 T cell population identified. Log 2 fold changes were calculated for the MFI of CD71 levels in the treated samples relative to the anti-CD3 stimulated controls. N=4 donors, 2 technical replicates±SEM.

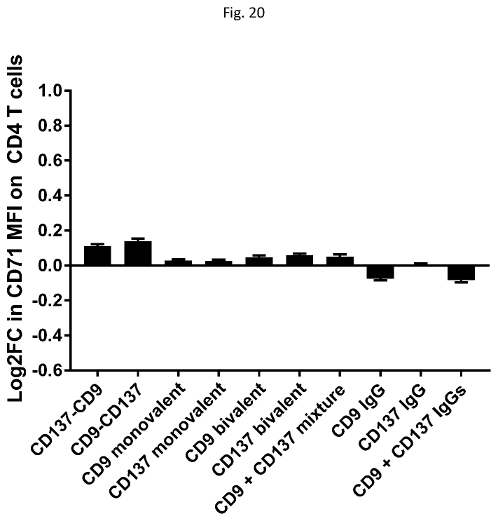

FIG. 21. Log 2 fold change in the MFI of CD71 expression on CD4 T cells in the absence of any stimulation. PBMC cultures were incubated for 48 hours in the presence of either the CD137-CD9 bispecific antibodies, control antibodies or corresponding IgG proteins. The samples were then analysed by flow cytometry and the CD4 T cell population identified. Log 2 fold changes were calculated for the MFI of CD71 levels in the treated samples relative to the untreated controls. N=4 donors, 2 technical replicates±SEM.

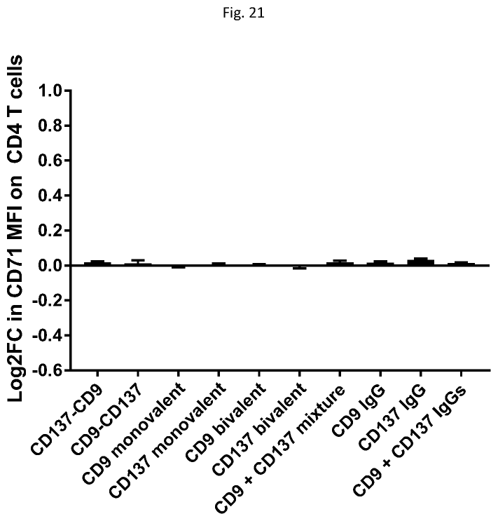

FIG. 22. Numbers of proliferating CD8+ and CD4+ T cells in the presence of anti-CD3 (long/mL) stimulation. Human PBMC were labelled with Cell Trace™ violet (CTV) then incubated in triplicate wells for 4 days with anti-CD3 plus 100 nM of the CD137-CD9 antibodies. T cell proliferation was assessed by flow cytometry by gating on CD8+(top plot) and CD4+(bottom plot) populations and enumeration of CTV-low cells. Results are presented as the mean±SEM. Similar data was obtained from a further 3 PBMC donors.

FIG. 23. Numbers of proliferating CD8+ and CD4+ T cells in the presence of anti-CD3 (10 ng/mL) stimulation. Human PBMC were labelled with Cell Trace™ violet (CTV) then incubated in triplicate wells for 4 days with anti-CD3 plus 100, 10 or 1 nM of the CD137-CD9 bispecific antibodies or control antibodies. T cell proliferation was assessed by flow cytometry by gating on CD8+(top plot) and CD4+(bottom plot) populations and enumeration of CTV-low cells. Results are presented as the mean±SEM. P means p-value or probability value (or significance value)—a value of p<0.05 or below suggests the observed difference is statistically significant.

FIG. 24. Log 2 fold change in the MFI of CD25 expression on CD8⁺ and CD4⁺ T cells in the presence of anti-CD3 (10 ng/mL) stimulation. Samples of the stimulated cultured PBMC were analysed upon treatment with 100 nM of FabX/FabY bispecific antibodies and full-length IgG CD137-CD9 bispecific antibody or bivalent and mixture controls, by flow cytometry, and the CD8⁺ and CD4⁺ T cell population identified. Log 2 fold changes were calculated for the MFI of CD25 levels in the treated samples relative to the anti-CD3 stimulated controls. N=4 donors, 4 technical replicates±SEM.

FIG. 25. Log 2 fold change in the MFI of CD71 expression on CD8⁺ and CD4⁺ T cells in the presence of anti-CD3 (10 ng/mL) stimulation. Samples of the stimulated cultured PBMC were analysed upon treatment with 100 nM of FabX/FabY bispecific antibodies and full-length IgG CD137-CD9 bispecific antibody or bivalent and mixture controls, by flow cytometry, and the CD8⁺ and CD4⁺ T cell population identified. Log 2 fold changes were calculated for the MFI of CD71 levels in the treated samples relative to the anti-CD3 stimulated controls. N=4 donors, 4 technical replicates±SEM.

FIG. 26. Log 2 fold change in the MFI of CD25 expression on CD8⁺ T cells in the presence of anti-CD3 (10 ng/mL) stimulation. Samples of the stimulated cultured PBMC were analysed upon treatment with 6 concentrations (300 nM maximum) of CD137-CD9 bispecific antibody or bivalent and mixture controls, by flow cytometry, and the CD8⁺ T-cell population identified. Log 2 fold changes were calculated for the MFI of CD25 levels in the treated samples relative to the anti-CD3 stimulated controls. N=4 donors, 4 technical replicates±SEM.

FIG. 27. Log 2 fold change in the MFI of CD25 expression on CD4⁺ T cells in the presence of anti-CD3 (10 ng/mL) stimulation. Samples of the stimulated cultured PBMC were analysed upon treatment with 6 concentrations (300 nM maximum) of CD137-CD9 bispecific antibody or bivalent and mixture controls, by flow cytometry, and the CD4⁺ T-cell population identified. Log 2 fold changes were calculated for the MFI of CD25 levels in the treated samples relative to the anti-CD3 stimulated controls. N=4 donors, 4 technical replicates±SEM.

FIG. 28. Log 2 fold change in the MFI of CD71 expression on CD8⁺ T cells in the presence of anti-CD3 (10 ng/mL) stimulation. Samples of the stimulated cultured PBMC were analysed upon treatment with 6 concentrations (300 nM maximum) of CD137-CD9 bispecific antibody or bivalent and mixture controls, by flow cytometry, and the CD8⁺ T-cell population identified. Log 2 fold changes were calculated for the MFI of CD71 levels in the treated samples relative to the anti-CD3 stimulated controls. N=4 donors, 4 technical replicates±SEM.

FIG. 29. Log 2 fold change in the MFI of CD71 expression on CD4⁺ T cells in the presence of anti-CD3 (10 ng/mL) stimulation. Samples of the stimulated cultured PBMC were analysed upon treatment with 6 concentrations (300 nM maximum) of CD137-CD9 bispecific antibody or bivalent and mixture controls, by flow cytometry, and the CD4⁺ T-cell population identified. Log 2 fold changes were calculated for the MFI of CD71 levels in the treated samples relative to the anti-CD3 stimulated controls. N=4 donors, 4 technical replicates±SEM.

FIG. 30. Log 2 fold change in the MFI of CD25 expression on CD8+ T-cells in the presence of anti-CD3 (10 ng/mL) stimulation. Samples of the stimulated PBMC were analysed upon treatment with CD137-CD9 Fab-K_{D}-Fab constructs by flow cytometry and the CD8+ T-cell population identified. Log 2 fold changes were calculated for the MFI of CD25 levels in the treated samples relative to the anti-CD3 stimulated controls. N=3 donors, 3 technical replicates±SEM.

FIG. 31. Log 2 fold change in the MFI of CD25 expression on CD8+ T-cells in the presence of anti-CD3 (10 ng/mL) stimulation. Samples of the stimulated PBMC were analysed upon treatment with CD137-CD9 Fab-K_{D}-Fab constructs by flow cytometry and the CD8+ T-cell population identified. Log 2 fold changes were calculated for the MFI of CD25 levels in the treated samples relative to the anti-CD3 stimulated controls. N=3 donors, 3 technical replicates±SEM.

FIG. 32. Log 2 fold change in the MFI of CD25 expression on CD8+ T-cells in the presence of anti-CD3 (10 ng/mL) stimulation. Samples of the stimulated PBMC were analysed upon treatment with CD9 and CD137 bivalent Fab-K_{D}-Fab constructs by flow cytometry and the CD8+ T-cell population identified. Log 2 fold changes were calculated for the MFI of CD25 levels in the treated samples relative to the anti-CD3 stimulated controls. N=3 donors, 3 technical replicates±SEM.

FIG. 33. Log 2 fold change in the MFI of CD25 expression on CD8+ T-cells in the presence of anti-CD3 (10 ng/mL) stimulation. Samples of the stimulated cultured PBMC were analysed upon treatment with CD9 and CD137 monovalent Fab-K_{D}-Fab constructs by flow cytometry and the CD8+ T-cell population identified. Log 2 fold changes were calculated for the MFI of CD25 levels in the treated samples relative to the anti-CD3 stimulated controls. N=3 donors, 3 technical replicates±SEM.

FIG. 34. Log 2 fold change in the MFI of CD25 expression on CD4+ T-cells in the presence of anti-CD3 (10 ng/mL) stimulation. Samples of the stimulated cultured PBMC were analysed upon treatment with CD137-CD9 Fab-K_{D}-Fab constructs by flow cytometry and the CD4+ T-cell population identified. Log 2 fold changes were calculated for the MFI of CD25 levels in the treated samples relative to the anti-CD3 stimulated controls. N=3 donors, 3 technical replicates±SEM.

FIG. 35. Log 2 fold change in the MFI of CD25 expression on CD4+ T-cells in the presence of anti-CD3 (10 ng/mL) stimulation. Samples of the stimulated cultured PBMC were analysed upon treatment with CD137-CD9 Fab-K_{D}-Fab constructs by flow cytometry and the CD4+ T-cell population identified. Log 2 fold changes were calculated for the MFI of CD25 levels in the treated samples relative to the anti-CD3 stimulated controls. N=3 donors, 3 technical replicates±SEM.

FIG. 36. Log 2 fold change in the median fluorescence intensity (MFI) of CD25 expression on CD4+ T-cells in the presence of anti-CD3 (10 ng/mL) stimulation. Samples of the stimulated cultured PBMC were analysed upon treatment with CD9 and CD137 bivalent Fab-K_{D}-Fab constructs by flow cytometry and the CD4+ T-cell population identified. Log 2 fold changes were calculated for the MFI of CD25 levels in the treated samples relative to the anti-CD3 stimulated controls. N=3 donors, 3 technical replicates±SEM.

FIG. 37. Log 2 fold change in the MFI of CD25 expression on CD4+ T-cells in the presence of anti-CD3 (10 ng/mL) stimulation. Samples of the stimulated cultured PBMC were analysed upon treatment with CD9 and CD137 monovalent Fab-K_{D}-Fab constructs by flow cytometry and the CD4+ T-cell population identified. Log 2 fold changes were calculated for the MFI of CD25 levels in the treated samples relative to the anti-CD3 stimulated controls. N=3 donors, 3 technical replicates±SEM.

FIG. 38. Binding profile of Fab-Y antibodies (10 μg/mL) to Fc-fusion CD137 peptides (P1-P6) corresponding to different combinations of cysteine-rich domains (CRDs). Data acquired using the Octet® RED384 System (FortéBio). A tick represents binding (greater than 0.05 nm from second baseline), NB represents no binding detected. Epitope mapping to each CRD was inferred from binding to the different peptides. Y represents a functional response based on the capacity to increase CD25 expression on T cells by greater than 0.5 Log 2 fold change in 3 donors when added as a Fab-K_{D}-Fab with anti-CD9. N represents no functional response. (Function data generated in Example 8).

FIG. 39. Log 2 fold change in the number of proliferating CD8+ and CD4+ T cells in the presence of anti-CD3 (50 ng/mL) stimulation. Human PBMC were labelled with Cell Trace™ violet (CTV) then incubated for 6 days with anti-CD3 plus 100 nM of the CD137-CD9 bispecific antibodies or control antibodies. T cell proliferation was assessed by flow cytometry by gating on CD8+(top plot) and CD4+(bottom plot) populations and enumeration of CTV-low cells. N=4 donors±SEM.

FIG. 40. Log 2 fold change in the MFI of CD71 on CD8+ and CD4+ T cells in the presence of anti-CD3 (50 ng/mL) stimulation. Human PBMC were incubated in triplicate wells for 6 days with anti-CD3 plus 100 nM of the CD137-CD9 bispecific antibodies or control antibodies. CD25 levels on the T cells was then measured by flow cytometry by gating on CD8+(top plot) and CD4+(bottom plot) populations. Log 2 fold changes were calculated for the MFI of CD71 levels in the treated samples relative to the anti-CD3 stimulated controls. N=4 donors±SEM.

FIG. 41. Log 2 fold change in the MFI of CD71 on CD8+ and CD4+ T cells in the presence of anti-CD3 (50 ng/mL) plus anti-CD28 (200 ng/mL) stimulation. Human PBMC were incubated for 6 days with anti-CD3 plus 100 nM of the CD137-CD9 bispecific antibodies or control antibodies. CD25 levels on the T cells was then measured by flow cytometry by gating on CD8+(top plot) and CD4+(bottom plot) populations. Log 2 fold changes were calculated for the MFI of CD71 levels in the treated samples relative to the anti-CD3 stimulated controls. N=4 donors±SEM.

FIG. 42. Log 2 fold change in the MFI of CD25 expression on CD8⁺ and CD4⁺ T cells in the presence of anti-CD3 (10 ng/mL) stimulation. Samples of the stimulated cultured PBMC were analysed upon treatment with 100 nM of Fab-K_{D}-Fab bispecific or BYbe™ antibodies by flow cytometry, and the CD8⁺ and CD4⁺ T cell population identified. Log 2 fold changes were calculated for the MFI of CD25 levels in the treated samples relative to the anti-CD3 stimulated controls. N=4 donors±SEM.

FIG. 43. Log 2 fold change in the MFI of CD71 expression on CD8⁺ and CD4⁺ T cells in the presence of anti-CD3 (10 ng/mL) stimulation. Samples of the stimulated cultured PBMC were analysed upon treatment with 100 nM of Fab-K_{D}-Fab bispecific or BYbe™ antibodies by flow cytometry, and the CD8⁺ and CD4⁺ T cell population identified. Log 2 fold changes were calculated for the MFI of CD71 levels in the treated samples relative to the anti-CD3 stimulated controls. N=4 donors±SEM.

FIG. 44. Melanoma patient PBMC were stimulated with melanoma peptide mix (1 μg/mL) for 6 days, in the presence or absence of IgG molecules (100 nM), CD137-Fab X/CD9-Fab Y (100 nM), or pembrolizumab (1 μg/mL). Unstimulated and peptide-stimulated vehicle controls (1% PBS) were also included. Following 6 days of culture, absolute concentrations of IFNγ and INFα were quantified in the conditioned medium by Luminex® bead assay and the remaining cells were stained for CD3, CD56 and CD71 marker expression, and CD3− CD56+ natural killer cells, analysed for CD71 activation marker expression by flow cytometry. Data presented as mean±SEM of n=4 or n=5 independent melanoma PBMC donors (cytokine and flow cytometry data respectively). Work performed at Celentyx, Birmingham, UK.

FIG. 45. BioMAP® profile of CD137-CD9 bispecific IgG in the StroHT29 and VascHT29 CRC panels. Individual IgG treatments are shown on the X-axis, and the Y-axis represents a Log 2-transformed fold change in the biomarker readouts for the IgG test samples (n=3) divided by vehicle controls (n≥6). The dashed lines indicate the 95% vehicle only controls significance range. CD137-CD9 bispecific IgG-mediated effects on IFNγ, TNF, IL-2 and collagen III in the StroHT29 model and IFN-γ in the VascHT29 model are summarised in each panel, denoted with the prefix Stro or Vasc respectively. Statistically significant changes in biomarker levels are indicated by an asterix, reflecting a >20% effect compared to the vehicle control (Log 2 fold change >0.20-0.33) and a p value <0.01 (unpaired T test). Work performed at Eurofins, Missouri, USA.

### DETAILED DESCRIPTION OF THE INVENTION

The present invention will now be described with respect to particular non-limiting aspects and embodiments thereof and with reference to certain figures and examples.

Technical terms are used by their common sense unless indicated otherwise. If a specific meaning is conveyed to certain terms, definitions of terms will be given in the context of which the terms are used.

Where the term “comprising” is used in the present description and claims, it does not exclude other elements. For the purposes of the present disclosure, the term “consisting of” is considered to be a preferred embodiment of the term “comprising of”.

Where an indefinite or definite article is used when referring to a singular noun, e.g. “an” or “the”, this includes a plural of that noun unless something else is specifically stated.

As used herein, the terms “treatment”, “treating” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Treatment thus covers any treatment of a disease in a mammal, particularly in a human, and includes: (a) preventing the disease from occurring in a subject, i.e. a human, which may be predisposed to the disease but has not yet been diagnosed as having it; (b) inhibiting the disease, i.e., arresting its development; and (c) relieving the disease, i.e., causing regression of the disease.

A “therapeutically effective amount” refers to the amount of antibody comprising the distinct antigen-binding portions binding CD137 and CD9 that, when administered to a mammal or other subject for treating a disease, is sufficient to affect such treatment for the disease.

The present invention provides for antibodies comprising a first antigen-binding portion binding CD137 and a second antigen-binding portion binding CD9. The first and the second antigen-binding portions are located in the same antibody, i.e. they are part of the same polypeptide chain and/or associate via one or more covalent and/or non-covalent associations (such as the screening format Fab-K_{D}-Fab described herein or the classic heavy and light chain association forming a full IgG antibody) or are covalently linked so as to form one single molecule (such as cross-linking two separately expressed polypeptide chains, optionally via specific cross-linking agents).

CD137 and in particular human CD137 (Uniprot accession number Q07011) is a T-cell costimulatory receptor induced on TCR activation (Nam et al. Curr. Cancer Drug Targets, 5:357-363 (2005)). It is also known by the names 4-1BB ligand receptor, T-cell antigen 4-1BB homolog, TNFRSF9 and T-cell antigen ILA. In addition to be expressed on activated CD4+ and CD8+ T-cells CD137 is also expressed on CD4+ and CD25+ regulatory T-cells, activated natural killer cells and other immune system cells such as monocytes, neutrophils and dendritic cells. The sequence of human CD137, including the signal peptide is shown as SEQ ID NO:1 (Table 1).

CD9 and in particular human CD9 (Uniprot accession number P21926) was discovered as a human B-lymphocyte differentiation antigen and it has been found to be widely expressed on many non-hematopoietic tissues including various cancer. It is also known as Tetraspanin-29, motility-related protein-1, 5H9 antigen, cell growth-inhibiting gene 2 protein, leukocyte antigen MIC3 and MRP-1 (Rappa et al., Oncotarget 6:10, 7970-7991 (2015)). CD9 is a tetraspanin that is broadly expressed in a variety of solid tissues and on a multitude of hematopoietic cells (Nature reviews Cancer (9) 40-55 (2009)). CD9 involvement has been shown in the invasiveness and tumorigenicity of human breast cancer cells (Oncotargets, 6:10 (2015)), the suppression of cell motility and metastasis (J. Exp. Med 177:5 (1993)) and to have a role in T cells activation (J. Exp. Med 184:2 (1994)).

CD9 has also been shown to be present in exosomes (Asia-Pac J Clin Oncol, 2018; 1-9). Exosomes are cell derived nanovesicles with size of 30-120 nm. The molecular composition of exosomes reflects their origin and include unique composition of tetraspanins. Exosomes are thoughts to constitute a potent mode of intercellular communication that is important in the immune response, cell-to-cell spread of infectious agents, and tumour progression.

The sequence of human CD9, including the signal peptide is shown as SEQ ID NO:2 (Table 1).

TABLE 1

| | |
|---|---|
| SEQ ID NO: 1 | MGNSCYNIVATLLLVLNFERTRSLQDPCSNCPAGTFCDNNRNQICSPC |
| Human CD137 | PPNSFSSAGGQRTCDICRQCKGVFRTRKECSSTSNAECDCTPGFHCL |
| | GAGCSMCEQDCKQGQELTKKGCKDCCFGTFNDQKRGICRPWTNCSL |
| | DGKSVLVNGTKERDVVCGPSPADLSPGASSVTPPAPAREPGHSPQI |
| | ISFFLALTSTALLFLLFFLTLRFSVVKRGRKKLLYIFKQPFMRPVQ |
| | TTQEEDGCSCRFPEEEEGGCEL |
| SEQ ID NO: 2 | MPVKGGTKCIKYLLFGFNFIFWLAGIAVLAIGLWLRFDSQTKSIFEQ |
| Human CD9 | ETNNNNSSFYTGVYILIGAGALMMLVGFLGCCGAVQESQCMLGLFFG |
| | FLLVIFAIEIAAAIWGYSHKDEVIKEVQEFYKDTYNKLKTKDEPQRE |
| | TLKAIHYALNCCGLAGGVEQFISDICPKKDVLETFTVKSCPDAIKEV |
| | FDNKFHIIGAVGIGIAVVMIFGMIFSMILCCAIRRNREMV |
| SEQ ID NO: 3 | GFSLSTYAMS |
| CDR-H1 | |
| VR8475 | |
| SEQ ID NO: 4 | AIWSGGTTDYTSWAKG |
| CDR-H2 | |
| VR8475 | |
| SEQ ID NO: 5 | MAVIFNAYTFDS |
| CDR-H3 | |
| VR8475 | |
| SEQ ID NO: 6 | QASQNIYSYLA |
| CDR-L1 | |
| VR8475 | |
| SEQ ID NO: 7 | GASTLAS |
| CDR-L2 | |
| VR8475 | |
| SEQ ID NO: 8 | QQGHSSGNVGNNV |
| CDR-L3 | |
| VR8475 | |
| SEQ ID NO: 9 | GFSLSSYAMG |
| CDR-H1 | |
| VR7272 | |
| SEQ ID NO: 10 | AIGSITATGYARWAKG |
| CDR-H2 | |
| VR7272 | |
| SEQ ID NO: 11 | EIYVGSAYAFDI |
| CDR-H3 | |
| VR7272 | |
| SEQ ID NO: 12 | QASQSISNYLA |
| CDR-L1 | |
| VR7272 | |
| SEQ ID NO: 13 | LASTLAS |
| CDR-L2 | |
| VR7272 | |
| SEQ ID NO: 14 | QQGYIDNVNKG |
| CDR-L3 | |
| VR7272 | |
| SEQ ID NO: 15 | QSVEESGGRLVTPGTPLTLTCTVSGFSLSTYAMSWVRQAPGKGLEWI |
| VH | GAIWSGGTTDYTSWAKGRFTISKASTTVDLKITSPTTEDTATYFCVR |
| | MAVIFNAYTFDSWGPGTLVTVSS |
| VR8475 | |
| SEQ ID NO: 16 | AAGCTTCGAAGCCACCATGGAGACTGGGCTGCGCTGGCTTCTCCTG |
| VH nucl. | GTCGCTGTGCTCAAAGGTGTCCAGTGTCAGTCGGTGGAGGAGTCC |
| VR8475 | GGGGGTCGCCTGGTCACGCCTGGGACACCCCTGACACTCACCTGC |
| | ACAGTCTCTGGATTCTCCCTCAGTACGTATGCAATGAGCTGGGTCC |
| | GCCAGGCTCCAGGGAAGGGGCTGGAGTGGATCGGAGCGATTTGGA |
| | GTGGTGGTACCACGGACTACACGAGCTGGGCGAAAGGCCGATTCA |
| | CCATCTCCAAAGCCTCGACCACGGTGGATCTGAAAATCACCAGTCC |
| | GACAACCGAGGACACGGCCACCTATTTCTGTGTCAGAATGGCAGTG |
| | ATTTTTAATGCTTACACTTTTGATTCCTGGGGCCCAGGCACCCTGG |
| | TCACCGTCTCGAGT |
| SEQ ID NO: 17 | AYDMTQTPSSVSAAVGGTVTIKCQASQNIYSYLAWYQQKPGQRPKL |
| VL | LIYGASTLASGVPSRFKGSGSGTDFTLTISDLECDDAATYYCQQGH |
| VR8475 | SSGNVGNNVFGGGTEVWK |
| SEQ ID NO: 18 | AAGCTTCGAAGCCACCATGAACATGAGGGCCCCCACTCAGCTGCTG |
| VL nucl. | GGGCTCCTGCTGCTCTGGCTCCCAGGTGCCAGATGTGCCTATGATA |
| VR8475 | TGACCCAGACTCCATCCTCCGTGTCTGCAGCTGTGGGAGGCACAGT |
| | CACCATCAAGTGCCAGGCCAGTCAGAACATTTACAGTTACTTAGCCT |
| | GGTATCAGCAGAAACCAGGGCAGCGTCCCAAGCTCCTGATCTATGG |
| | TGCGTCCACTCTGGCATCTGGGGTCCCATCGCGGTTCAAAGGCAG |
| | TGGATCTGGGACAGATTTCACTCTCACCATCAGCGACCTGGAGTGT |
| | GACGATGCTGCCACTTACTACTGTCAACAGGGCCATAGTAGTGGTA |
| | ATGTTGGAAATAATGTTTTCGGCGGAGGGACCGAGGTGGTGGTCAA |
| | AGGTACG |
| SEQ ID NO: 19 | QSLEESGGRLVTPGTPLTLTCTVSGFSLSSYAMGWVRQAPGKGLE |
| VH | WIGAIGSITATGYARWAKGRFSISKTSTTVDLKMTSPTTEDTATY |
| VR7272 | FCAREIYVGSAYAFDIWGPGTLVTVSS |
| SEQ ID NO: 20 | AAGCTTCGAAGCCACCATGGAGACTGGGCTGCGCTGGCTTCTCCTG |
| VH nucl. | GTCGCTGTGCTCAAAGGTGTCCAGTGTCAGTCGCTGGAGGAGTCC |
| VR7272 | GGGGGTCGCCTGGTCACGCCTGGGACACCCCTGACACTCACCTGC |
| | ACAGTCTCTGGATTCTCCCTCAGTAGCTATGCAATGGGCTGGGTCC |
| | GCCAGGCTCCAGGGAAGGGGCTGGAGTGGATCGGAGCCATTGGTA |
| | GTATTACTGCCACTGGCTACGCGCGCTGGGCAAAAGGCCGATTCAG |
| | CATCTCCAAGACCTCGACCACGGTGGATCTGAAAATGACCAGTCCG |
| | ACAACCGAGGACACGGCCACCTATTTCTGTGCCAGAGAGATTTATG |
| | TTGGGTCTGCTTATGCCTTTGACATCTGGGGCCCAGGCACCCTGGT |
| | CACCGTCTCGAGT |
| SEQ ID NO: 21 | AYDMTQTPASVEVAVGDTVTIKCQASQSISNYLAWYQQKPGQPPKL |
| VL | LIYLASTLASGVPSRFKGSGSGTEFTLTISDLECADAATYYCQQGY |
| VR7272 | IDNVNKGFGGGTEWVK |
| SEQ ID NO: 22 | AAGCTTCGAAGCCACCATGGACACGAGGGCCCCCACTCAGCTGCT |
| VL nucl. | GGGGCTCCTGCTGCTCTGGCTCCCAGGTGCCAGATGTGCCTATGAT |
| VR7272 | ATGACCCAGACTCCAGCCTCTGTGGAGGTAGCTGTGGGAGACACTG |
| | TCACCATCAAGTGTCAGGCCAGTCAGAGCATTAGTAACTACTTAGCC |
| | TGGTATCAGCAGAAACCAGGGCAGCCTCCCAAGCTCCTGATCTATC |
| | TGGCATCTACTCTGGCATCTGGGGTCCCATCGCGGTTCAAAGGCAG |
| | TGGATCTGGGACAGAGTTCACTCTCACCATCAGCGACCTGGAGTGT |
| | GCCGATGCTGCCACTTACTATTGTCAACAGGGTTATATTGATAATGT |
| | TAATAAAGGTTTCGGCGGAGGGACCGAGGTGGTGGTCAAACGTAC |
| | G |

Within the present invention, unless recited otherwise, human CD137 and CD9 are always intended to be included in the term “CD137” and CD9″. However, unless “human CD137” and/or “human CD9” are explicitly used, the terms “CD137” and/or “CD9” include the same targets in other species, especially non-primate (e.g. rodents) and non-human primate (such as cynomolgus monkey) species.

The present invention therefore provides for an antibody comprising a first antigen-binding portion binding human CD137 and a second antigen-binding portion binding human CD9. The first and the second antigen-binding portions are located on the same antibody, i.e. they are part of the same polypeptide chain, they associate via one or more non-covalent and/or covalent associations or are linked so as to form one single molecule.

The present invention also provides for an antibody comprising a first antigen-binding portion binding an extracellular domain region of human CD137 and a second antigen-binding portion binding an extracellular domain region of human CD9. The first and the second antigen-binding portions are located on the same antibody, i.e. they are part of the same polypeptide chain, they associate via one or more non-covalent and/or covalent associations or are linked so as to form one single molecule.

More specifically, there is provided for an antibody comprising a first antigen-binding portion binding human CD137 as defined in SEQ ID NO: 1 or from amino acid 24 to 186 of SEQ ID NO: 1 alternatively from amino acid 25 to 186 or wherein preferably the first antigen-binding portion binds within amino acids 25 to 68 of SEQ ID NO: 1, more preferably within amino acids 25 to 45 of SEQ ID NO: 1 and a second antigen-binding portion binding human CD9 as defined in SEQ ID NO: 2 or from amino acid 2 to 228 of SEQ ID NO: 2, alternatively from amino acid 34 to 55 of SEQ ID NO: 2 or preferably from the second antigen-binding portion binds within amino acid 112-195. The first and the second antigen-binding portions are located on the same antibody, i.e. they are part of the same polypeptide chain, they associate via one or more non-covalent and/or covalent associations or are linked so as to form one single molecule.

Antibodies against CD137 have been disclosed in several US granted patents such as U.S. Pat. No. 7,288,638 or U.S. Pat. No. 6,887,673. Urelumab is an anti-CD137 fully human IgG4 monoclonal antibody currently in the clinic. In one embodiment, the present invention discloses an antibody comprising a first antigen-binding portion binding human CD137, which is the antigen-binding portion of urelumab, and a second antigen-binding portion binding human CD9.

The monoclonal antibody of the present invention, upon binding of CD137 and CD9, stimulates T-cell activation, i.e. further activates T-cells and enhances induction of T-cell proliferation, and in particular, the monoclonal antibody comprising a first antigen-binding portion binding CD137 and a second antigen-binding portion biding CD9 further activates T-cells and enhances induction of T-cell proliferation in the presence of an anti-CD3 stimulation. More specifically, the monoclonal antibody comprising a first antigen-binding portion binding CD137 and a second antigen-binding portion binding CD9 further activates T-cells and enhances induction of T-cell proliferation in the presence of an anti-CD3 stimulation but it does not activate unstimulated T-cells. More specifically, the T-cell is at least a CD4+ T-cell or at least a CD8+ T-cell or a mixture thereof.

The term “activate” (and grammatical variations thereof) as used herein at least includes the upregulation of specific T-cell markers, i.e. increased transcription and/or translation of these markers and/or trafficking of these newly transcribed/translated markers or of any marker already expressed to the cell membrane, and the induction of proliferation.

Hence, the present invention provides for a monoclonal antibody comprising a first antigen-binding portion binding CD137 and a second antigen-binding portion binding CD9 capable of activating T-cells in the presence of an anti-CD3 stimulation wherein the further activation of T-cell is measured as an upregulation of T-cell markers and the enhancement of T cell proliferation.

Specific markers of T-cell activation and proliferation include but are not limited to the upregulation of CD25, CD71 and CD137.

In one preferred embodiment of the present invention, the monoclonal antibody comprising a first antigen-binding portion binding CD137 and a second antigen-binding portion binding CD9 is capable of activating T-cells in the presence of an anti-CD3 stimulation wherein activating T-cells results in an upregulation of CD71, CD25 and CD137.

The term “antibody” as used herein includes whole immunoglobulin molecules and antigen-binding portions of immunoglobulin molecules associated via non-covalent and/or covalent associations or linked together, optionally via a linker.

In one embodiment, the antigen-binding portions binding CD137 and CD9 are the antigen-binding portions of an IgG, wherein one arm binds CD137 and the other arm binds CD9.

In another embodiment, the antigen-biding portions comprised in the antibody are functionally active fragments or derivatives of a whole immunoglobulin and may be, but are not limited to, VH, VL, VHH, Fv, scFv fragment (including dsscFv), Fab fragments, modified Fab fragments, Fab′ fragments, F(ab′)₂ fragments, Fv and epitope-binding fragments of any of the above.

Other antibody fragments include those described in WO2005003169, WO2005003170, WO2005003171, WO2009040562 and WO2010035012. Functionally active fragments or derivative of a whole immunoglobulin and methods of producing them are well known in the art, see for example Verma et al., 1998, Journal of Immunological Methods, 216, 165-181; Adair and Lawson, 2005. Therapeutic antibodies. Drug Design Reviews—Online 2(3):209-217.

In one embodiment of the invention each of the antigen-binding portions is independently selected from a Fab, a Fab′, a scFv or a VHH. In one embodiment, the antigen-binding portion binding CD137 is a Fab whilst the antigen-binding portion binding CD9 is a scFv. In another embodiment, the antigen-binding portion binding CD9 is a Fab whilst the antigen-binding portion binding CD137 is a scFv. In another embodiment, both antigen-binding portions are a Fab or scFv.

In one preferred embodiment, the antibody is monoclonal, which means that the antigen-binding portions comprised therein are all monoclonal. Therefore, in one preferred embodiment of the present invention, there is provided a monoclonal antibody comprising a first antigen-binding portion binding CD137 and a second antigen-binding portion binding CD9. Preferably, this antibody is capable of further activating T-cells and/or enhancing induction of T-cell proliferation in the presence of an anti-CD3 stimulation wherein activating T-cell results in an upregulation of CD71, CD25 and CD137.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by for example the methods described by Babcook, J. et al, 1996, Proc. Natl. Acad. Sci. USA 93(15):7843-78481; WO92/02551; WO2004/051268 and WO2004/106377.

The antibodies of the present invention can also be generated using various phage display methods known in the art and include those disclosed by Brinkman et al. (in J. Immunol. Methods, 1995, 182: 41-50), Ames et al. (J. Immunol. Methods, 1995, 184: 177-186), Kettleborough et al. (Eur. J. Immunol. 1994, 24:952-958), Persic et al. (Gene, 1997 187 9-18), Burton et al. (Advances in Immunology, 1994, 57: 191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. When the antigen-binding portions comprised in the antibody are functionally active fragments or derivatives of a whole immunoglobulin such as single chain antibodies, they may be made such as those described in U.S. Pat. No. 4,946,778 which can also be adapted to produce single chain antibodies binding to CD137 and CD9. Transgenic mice, or other organisms, including other mammals, may be used to express antibodies, including those within the scope of the invention.

The antibody of the present invention may be chimeric, human or humanised.

Chimeric antibodies are those antibodies encoded by immunoglobulin genes that have been genetically engineered so that the light and heavy chain genes are composed of immunoglobulin gene segments belonging to different species.

Humanized, antibodies are antibody molecules from non-human species having one or more complementarity determining regions (CDRs) from the non-human species and a framework region from a human immunoglobulin molecule (see, e.g. U.S. Pat. No. 5,585,089; WO91/09967). Preferably the antibody of the present invention is humanized. In one embodiment of the present invention, there is provided an antibody, preferably a monoclonal antibody, comprising a first antigen-binding portion binding CD137 and a second antigen-binding portion binding CD9, wherein the antibody is humanised. More preferably this antibody is capable of activating T-cells and/or enhancing induction of T-cell proliferation in the presence of an anti-CD3 stimulation wherein activating T-cells results in an upregulation of CD71, CD25 and CD137.

In humanized antibodies, the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment, rather than the entire CDR being transferred, only one or more of the specificity determining residues from any one of the CDRs described herein above are transferred to the human antibody framework (see, for example, Kashmiri et al., 2005, Methods, 36, 25-34). When the CDRs or specificity determining residues are grafted, any appropriate acceptor variable region framework sequence may be used having regard to the class/type of the donor antibody from which the CDRs are derived, including mouse, primate and human framework regions. Preferably, the humanized antibody according to the invention comprises a variable domain comprising human acceptor framework regions as well as one or more of the CDRs or specificity determining residues described above. Thus, provided in one embodiment is a humanized monoclonal antibody comprising an antigen-binding portion binding CD137 and an antigen-binding portion binding CD9, wherein each antigen-binding portion comprises a variable domain comprising human acceptor framework regions and non-human donor CDRs.

Examples of human frameworks which can be used in the invention are KOL, NEWM, REI, EU, TUR, TEI, LAY and POM (Kabat et al, supra). For example, KOL and NEWM can be used for the heavy chain, REI can be used for the light chain and EU, LAY and POM can be used for both the heavy chain and the light chain. Alternatively, human germline sequences may be used; these are available at, for example: http://vbase.mrc-cpe.cam.ac.uk/. In a CDR-grafted antibody of the invention, the acceptor heavy and light chains do not necessarily need to be derived from the same antibody and may, if desired, comprise composite chains having framework regions derived from different chains.

Fully human antibodies are those antibodies in which the variable regions and the constant regions (where present) of both the heavy and the light chains are all of human origin, or substantially identical to sequences of human origin, not necessarily from the same antibody. Examples of fully human antibodies may include antibodies produced for example by the phage display methods described above and antibodies produced by mice in which the murine immunoglobulin variable and constant region genes have been replaced by their human counterparts, e.g., as described in general terms in EP0546073, U.S. Pat. Nos. 5,545,806, 5,569,825, 5,625,126, 5,633,425, 5,661,016, 5,770,429, EP 0438474 and EP0463151.

Furthermore, the antibody of the invention may comprise a heavy chain constant region selected from an IgG1, an IgG2, an IgG3 or an IgG4 isotype, or a variant thereof. The constant region domains of the antibody of the invention, if present, may be selected having regard to the proposed function of the antibody, and in particular the effector functions which may be required. For example, the human IgG constant region domains of the IgG1 and IgG3 isotypes may be used when the antibody effector functions are required. Alternatively, IgG2 and IgG4 isotypes may be used when the antibody effector functions are not required. For example, IgG4 molecules in which the serine at position 241 has been changed to proline as described in Angal et al., Molecular Immunology, 1993, 30 (1), 105-108, may be used. Particularly preferred is the IgG4 constant domain that comprises this change.

It should also be appreciated that antigen-binding portions comprised in the antibody of the invention such as the functionally-active fragments or derivatives of whole immunoglobulin fragments described above, may be incorporated into other antibody formats than being the antigen-binding portions of the classic IgG format. Alternative format to the classic IgG may include those known in the art and those described herein, such as DVD-Igs, FabFvs for example as disclosed in WO2009/040562 and WO2010/035012, diabodies, triabodies, tetrabodies etc. Other examples include a diabody, triabody, tetrabody, bibodies and tribodies (see for example Holliger and Hudson, 2005, Nature Biotech 23(9): 1 126-1136; Schoonjans et al. 2001, Biomolecular Engineering, 17 (6), 193-202), tandem scFv, tandem scFv-Fc, FabFv, Fab′Fv, FabdsFv, Fab-scFv, Fab′-scFv, diFab, diFab′, scdiabody, scdiabody-Fc, ScFv-Fc-scFv, scdiabody-CH3, IgG-scFv, scFv-IgG, V-IgG, IgG-V, DVD-Ig, DuoBody, Fab-Fv-Fv, Fab-Fv-Fc and Fab-dsFv-PEG fragments described in WO2009040562, WO2010035012, WO2011/08609, WO2011/030107 and WO201 1/061492, respectively.

Furthermore, the antibody of the invention may comprise along with the antigen-binding portions binding CD137 and CD9, also at least an additional antigen-binding portion. Therefore, in one embodiment, there is provided an antibody, preferably a monoclonal antibody, comprising a first antigen-binding portion binding CD137 and a second antigen-binding portion binding CD9, wherein the antibody is humanised and wherein the antibody further comprises an additional antigen-binding portion. More preferably this antibody is capable of activating T-cells and/or enhancing induction of T-cell proliferation. The stimulation may be an anti-CD3 stimulation wherein activating T-cells may result in an upregulation of CD71, CD25 and CD137.

In one embodiment, the additional antigen-binding portion is capable of increasing, i.e. extending, the half-life of the antibody. Preferably, the additional antigen-binding portion binds albumin, more preferably human serum albumin.

In one preferred embodiment, the antibody comprises a first antigen-binding portion binding the CRD1 domain of human CD137, wherein preferably the first antigen-binding portion binds within amino acids 25 to 68 of SEQ ID NO: 1, more preferably within amino acids 25 to 45 of SEQ ID NO: 1 and a second antigen-binding portion binding an extracellular domain region of human CD9, wherein the extracellular domain region of CD9 is preferably loop 2 of CD9, and wherein more preferably the second antigen-binding portion binds within amino acids 112 to 195 of SEQ ID NO: 2, wherein the first and the second antigen-binding portions are located on the same antibody, i.e. they are part of the same polypeptide chain, associate via one or more non-covalent and/or covalent associations or linked so as to form one single molecule.

In another embodiment, the first antigen-binding portion binding CD137 of the antibody of the present invention comprises a first heavy chain variable region and a first light chain variable region and the second antigen-binding portion binding CD9 comprises a second heavy chain variable region and a second light chain variable region and wherein:
a. The first heavy chain variable region comprises a CDR-H1 comprising SEQ ID NO: 3, a CDR-H2 comprising SEQ ID NO: 4 and a CDR-H3 comprising SEQ ID NO: 5; and
b. The first light chain variable region comprises a CDR-L1 comprising SEQ ID NO: 6, a CDR-L2 comprising SEQ ID NO: 7 and a CDR-L3 comprising SEQ ID NO: 8; and
c. The second heavy chain variable region comprises a CDR-H1 comprising SEQ ID NO: 9, a CDR-H2 comprising SEQ ID NO: 10 and a CDR-H3 comprising SEQ ID NO: 11; and
d. The second light chain variable region comprises a CDR-L1 comprising SEQ ID NO: 12, a CDR-L2 comprising SEQ ID NO: 13 and a CDR-L3 comprising SEQ ID NO: 14;
or
e. The first heavy chain variable region comprises SEQ ID NO: 15 and the first light chain variable region comprises SEQ ID NO: 17; and the second heavy chain variable region comprises SEQ ID NO: 19 and second light chain variable region comprises SEQ ID NO: 21;
or
f. The first heavy chain variable region is encoded by a nucleotide sequence comprising SEQ ID NO: 16 and the first light chain variable region is encoded by a nucleotide sequence comprising SEQ ID NO: 18; and the second heavy chain variable region is encoded by a nucleotide sequence comprising SEQ ID NO: 20 and second light chain variable region is encoded by a nucleotide sequence comprising SEQ ID NO: 22.

In one embodiment, the antibody according to the present invention is prepared according to the disclosure of WO2015/181282, WO2016/009030, WO2016/009029, WO2017/093402, WO2017/093404 and WO2017/093406, which are all incorporated herein by reference.

More specifically, the antibody is made by the heterodimerization of a Fab-X and a Fab-Y.

Fab-X comprises a Fab fragment which comprises the first antigen-binding portion binding CD137 which comprises a first heavy chain variable region and a first light chain variable region wherein the first heavy chain variable region comprises a CDR-H1 comprising SEQ ID NO: 3, a CDR-H2 comprising SEQ ID NO: 4 and a CDR-H3 comprising SEQ ID NO: 5; and the first light chain variable region comprises a CDR-L1 comprising SEQ ID NO: 6, a CDR-L2 comprising SEQ ID NO: 7 and a CDR-L3 comprising SEQ ID NO: 8. The Fab comprising the first antigen-binding portion binding CD137 is linked to a scFv (clone 52SR4), preferably via a peptide linker to the C-terminal of the CH1 domain of the Fab fragment and the VL domain of the scFv. The scFv may itself also contains a peptide linker located in between its VL and VH domains.

Fab-Y also comprises a Fab fragment which comprises the second antigen-binding portion binding CD9 which comprises a second heavy chain variable region and a second light chain variable region and wherein the second heavy chain variable region comprises a CDR-H1 comprising SEQ ID NO: 9, a CDR-H2 comprising SEQ ID NO: 10 and a CDR-H3 comprising SEQ ID NO: 11; and the second light chain variable region comprises a CDR-L1 comprising SEQ ID NO: 12, a CDR-L2 comprising SEQ ID NO: 13 and a CDR-L3 comprising SEQ ID NO: 14. The Fab comprising the second antigen-binding portion binding CD9 is linked to a peptide GCN4 (clone 7P14P), preferably via a peptide linker to the CH1 domain of the Fab fragment.

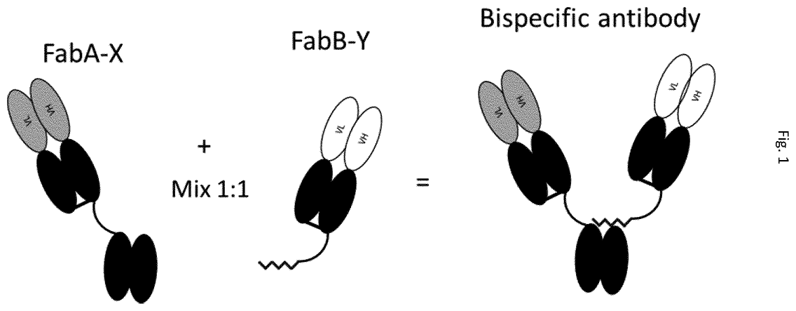

The scFv of Fab-X is specific for and complementary to the peptide GCN4 of Fab-Y. As a result, when the Fab-X and the Fab-Y are brought into contact with each other, a non-covalent binding interaction between the scFv and GCN4 peptide occurs, thereby physically retaining the two antigen-binding portions in the form of a complex resulting in an antibody comprising two antigen-binding portions on the same molecule (FIG. 1).

In another embodiment, the Fab-X comprises the first antigen-binding portion binding CD137 which comprises a first heavy chain variable region comprising SEQ ID NO: 15 and the first light chain variable region comprising SEQ ID NO: 17; and Fab-Y comprises the second antigen-binding portion binding CD9 which comprises the second heavy chain variable region comprising SEQ ID NO: 19 and second light chain variable region comprising SEQ ID NO: 21.

Binding specificities may be exchanged between Fab-X and Fab-Y, i.e. in one embodiment Fab-X may comprise the antigen-binding portion binding to CD137 or the antigen-binding portion binding to CD9 and vice-versa, Fab-Y may comprise the antigen-binding portion binding to CD137 or the antigen-binding portion binding to CD9.

The antibody of the present invention may be comprised in a pharmaceutical composition along with one or more pharmaceutically acceptable excipients. By pharmaceutical composition is intended a composition for both therapeutic and diagnostic use. In another aspect, the present invention provides for a pharmaceutical composition comprising an antibody, preferably a monoclonal antibody, comprising a first antigen-binding portion binding CD137 and a second antigen-binding portion binding CD9, wherein the antibody is preferably humanised and wherein the composition comprises one or more pharmaceutically acceptable excipients.

Pharmaceutically acceptable excipients in therapeutic compositions may additionally contain liquids such as water, saline, glycerol and ethanol. Additionally, auxiliary substances, such as wetting or emulsifying agents or pH buffering substances, may be present in such compositions. Such excipients enable the pharmaceutical compositions to be formulated as tablets, pills, dragees, capsules, liquids, gels, syrups, slurries and suspensions, for ingestion by the patient.

The antibody of the present invention and the pharmaceutical composition comprising such antibody may be used in therapy.

Therefore, in another aspect, the present invention provides for an antibody, preferably a monoclonal antibody, or a pharmaceutical composition comprising the antibody, and one or more pharmaceutically acceptable excipients, wherein the antibody comprises a first antigen-binding portion binding CD137 and a second antigen-binding portion binding CD9, wherein the antibody is preferably humanised and is for use in therapy.

In one embodiment, the antibody or composition comprising such antibody for use in therapy is an antibody comprising a first antigen-binding portion binding the CRD1 domain of human CD137, wherein preferably the first antigen-binding portion binds within amino acids 25 to 68 of SEQ ID NO: 1, more preferably within amino acids 25 to 45 of SEQ ID NO: 1 and a second antigen-binding portion binding an extracellular domain region of human CD9, wherein the extracellular domain region of CD9 is preferably loop 2 of CD9, and wherein more preferably the second antigen-binding portion binds within amino acids 112 to 195 of SEQ ID NO: 2, wherein the first and the second antigen-binding portions are located on the same antibody, i.e. they are part of the same polypeptide chain, associate via one or more non-covalent and/or covalent associations or linked so as to form one single molecule.

In another aspect, the present invention provides for an antibody, preferably a monoclonal antibody, or a pharmaceutical composition comprising the antibody, and one or more pharmaceutically acceptable excipients, wherein the antibody comprises a first antigen-binding portion binding CD137 and a second antigen-binding portion binding CD9, wherein the antibody is preferably humanised and is for use in the treatment of cancer and/or an infectious disease.

In another embodiment, the antibody or composition comprising such antibody for use in the treatment of cancer and/or an infectious disease is an antibody comprising a first antigen-binding portion binding the CRD1 domain of human CD137, wherein preferably the first antigen-binding portion binds within amino acids 25 to 68 of SEQ ID NO: 1, more preferably within amino acids 25 to 45 of SEQ ID NO: 1 and a second antigen-binding portion binding an extracellular domain region of human CD9, wherein the extracellular domain region of CD9 is preferably loop 2 of CD9, and wherein more preferably the second antigen-binding portion binds within amino acids 112 to 195 of SEQ ID NO: 2, wherein the first and the second antigen-binding portions are located on the same antibody, i.e. they are part of the same polypeptide chain, associate via one or more non-covalent and/or covalent associations or linked so as to form one single molecule.

In yet another aspect, the present invention provides for a method for treating a subject afflicted with cancer and/or an infectious disease, comprising administering to the subject a pharmaceutically effective amount of an antibody, preferably a monoclonal antibody, or a pharmaceutical composition comprising the antibody, and one or more pharmaceutically acceptable excipients, wherein the antibody comprises a first antigen-binding portion binding CD137 and a second antigen-binding portion binding CD9, wherein the antibody is preferably humanised.

The subjects to be treated is preferably a human subject. In one embodiment, there is provided for a method for treating a human subject afflicted with cancer and/or an infectious disease, comprising administering to the subject a pharmaceutically effective amount of an antibody, preferably a monoclonal antibody, or a pharmaceutical composition comprising the antibody and one or more pharmaceutically acceptable excipients, wherein the antibody comprises a first antigen-binding portion binding CD137 and a second antigen-binding portion binding CD9, wherein the antibody is preferably humanised.

In yet another embodiment, the method for treating a human subject afflicted with cancer and/or an infectious disease comprises administering an antibody or composition comprising such antibody comprising a first antigen-binding portion binding the CRD1 domain of human CD137, wherein preferably the first antigen-binding portion binds within amino acids 25 to 68 of SEQ ID NO: 1, more preferably within amino acids 25 to 45 of SEQ ID NO: 1 and a second antigen-binding portion binding an extracellular domain region of human CD9, wherein the extracellular domain region of CD9 is preferably loop 2 of CD9, and wherein more preferably the second antigen-binding portion binds within amino acids 112 to 195 of SEQ ID NO: 2, wherein the first and the second antigen-binding portions are located on the same antibody, i.e. they are part of the same polypeptide chain, associate via one or more non-covalent and/or covalent associations or linked so as to form one single molecule.

In another aspect of the present invention, there is provided the use of an antibody, preferably a monoclonal antibody, or a pharmaceutical composition comprising the antibody, and one or more pharmaceutically acceptable excipients, wherein the antibody comprises a first antigen-binding portion binding CD137 and a second antigen-binding portion binding CD9, wherein the antibody is preferably humanised, in the manufacture of a medicament for treating cancer and/or an infectious disease.

Example of cancers that may be treated using the antibody, or pharmaceutical composition comprising such antibody, include but are not limited to, Acute Lymphoblastic Leukaemia, Acute Myeloid Leukaemia, Adrenocortical Carcinoma, AIDS-Related Cancers Kaposi Sarcoma (Soft Tissue Sarcoma), AIDS-Related Lymphoma, Primary CNS Lymphoma, Anal Cancer, Appendix Cancer, Astrocytomas, Atypical Teratoid/Rhabdoid Tumour, Brain Cancer, Basal Cell Carcinoma of the Skin, Bile Duct Cancer, Bladder Cancer, Bone Cancer (includes Ewing Sarcoma and Osteosarcoma and Malignant Fibrous Histiocytoma), Breast Cancer, Bronchial Tumours, Burkitt Lymphoma, Carcinoid Tumour, Cardiac (Heart) Tumours, Embryonal Tumours, Germ Cell Tumour, Primary CNS Lymphoma, Cervical Cancer, Cholangiocarcinoma, Chordoma, Chronic Lymphocytic Leukaemia, Chronic Myelogenous Leukaemia, Chronic Myeloproliferative Neoplasms, Colorectal Cancer, Craniopharyngioma, Cutaneous T-Cell Lymphoma, Ductal Carcinoma In Situ, Endometrial Cancer (Uterine Cancer), Ependymoma, Oesophageal Cancer, Esthesioneuroblastoma (Head and Neck Cancer), Ewing Sarcoma (Bone Cancer), Extracranial Germ Cell Tumour, Extragonadal Germ Cell Tumour, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumour, Gastrointestinal Stromal Tumours (Soft Tissue Sarcoma), Ovarian Germ Cell Tumours, Testicular Cancer, Gestational Trophoblastic Disease, Hairy Cell Leukaemia, Head and Neck Cancer, Hepatocellular (Liver) Cancer, Histiocytosis, Langerhans Cell Hodgkin Lymphoma, Hypopharyngeal Cancer, Intraocular Melanoma, Islet Cell Tumours, Pancreatic Neuroendocrine Tumours, Kaposi Sarcoma (Soft Tissue Sarcoma), Kidney (Renal Cell) Cancer, Langerhans Cell Histiocytosis, Laryngeal Cancer, Leukaemia, Lip and Oral Cavity Cancer, Liver Cancer, Lung Cancer (Non-Small Cell and Small Cell), Male Breast Cancer, Malignant Fibrous Histiocytoma of Bone and Osteosarcoma, Melanoma, Intraocular (Eye) Childhood Intraocular, Merkel Cell Carcinoma, Mesothelioma, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukaemia, Chronic, Myeloid Leukaemia, Acute, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumours (Islet Cell Tumours), Papillomatosis, Paraganglioma, Paranasal Sinus and Nasal Cavity Cancer, Parathyroid Cancer, Penile Cancer, Pharyngeal Cancer, Pheochromocytoma, Pituitary Tumour, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma Childhood Rhabdomyosarcoma, Childhood Vascular Tumours, Ewing Sarcoma, Kaposi Sarcoma, Osteosarcoma, Soft Tissue Sarcoma, Uterine Sarcoma, Sézary Syndrome, Skin Cancer, Small Intestine Cancer, Squamous Cell Carcinoma of the Skin, Squamous Neck Cancer with Occult Primary, Stomach (Gastric) Cancer, Cutaneous T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Urethral Cancer, Uterine Cancer, Endometrial Uterine Sarcoma, Vaginal Cancer, Vascular Tumours, Vulvar Cancer and Wilms Tumour, and any combinations of these cancers. The present invention is also applicable to treatment of metastatic cancers.

The antibody according to the present invention, or the pharmaceutical composition comprising such antibody, may be administered concomitantly or sequentially to one or more additional cancer therapies. By cancer therapies is intended drug based therapies as well as other type of cancer therapies such as radiotherapies.

The invention will now be further described by way of examples with references to embodiments illustrated in the accompanying drawings

### EXAMPLES

### Example 1: T-Cell Activation Primary Screen

The activation status of T cells can be assessed through their expression of cell surface markers and secreted cytokines which play important roles in cellular function. Activated T cells for example upregulate CD25, CD71 and CD137 on their surface. T cells in the tumour microenvironment are often maintained in a suppressed state and express only low levels of these proteins. Agents that overcome this suppression and induce T cell activation and proliferation have tremendous therapeutic potential as they may unleash effective anti-tumour T cell responses and promote cancer elimination.

Various phenotypes such as the enhancement of activated T-cells through inhibition of T-cell expressed markers, upregulation of cytokine release and T-cell proliferation were investigated in order to reflect different biological mechanisms important for the therapy of cancer and infectious diseases.

To identify novel modulators of T cell activation, a large screen was undertaken whereby 49 antigen specificities were combined to generate a grid of bispecific antibodies with a theoretical size of 1,176 possible bispecific combinations. The specificities were selected from the literature as being expressed on T cells or expressed on other cell types involved in interactions with T cells. Of this potential grid, 969 novel antigen bispecific constructs were tested, covering 82.4% of the possible combinations. Between 1 and 4 different antibodies were tested for each specificity, and all were tested on peripheral blood mononuclear cells (PBMC) from donors in combination with a negative control arm to identify the effect of the monovalent forms of the construct.

PBMC represent the major leukocyte classes involved in both innate and adaptive immunity, apart from granulocytes. PBMC comprise a heterogenous population of cells which when manipulated in vitro provide a relatively more relevant physiological environment compared to isolated component cell types such as T cells and monocytes, that are no longer capable of responding to paracrine and autocrine signals provided by other cells. As such identification of molecules modulating specific subsets of cells within the wider PBMC population, have increased translational potential to more complex biological systems, ultimately increasing success rates for modulating immune cell interactions in disease.

Each bispecific combination was tested on two PBMC donors. This negative control arm is a Fab from an antibody raised to an antigen not expressed on PBMC.

Fusion proteins were prepared according to the disclosure of WO2015/181282, WO2016/009030, WO2016/009029, WO2017/093402, WO2017/093404 and WO2017/093406, which are all incorporated herein by reference. The first fusion protein (A-X) includes a Fab fragment (A of the A-X) with specificity to one antigen, which is linked to X, a scFv (clone 52SR4) via a peptide linker to the C-terminal of the CH1 domain of the Fab fragment and the VL domain of the scFv. The scFv itself also contains a peptide linker located in between its VL and VH domains.

The second fusion protein (B-Y) includes a Fab fragment (B of the B-Y) with specificity to another antigen. However, in comparison to the first protein, the Fab fragment B is attached to Y, a peptide GCN4 (clone 7P14P) via a peptide linker to the CH1 domain of the Fab fragment.

The scFv, X, is specific for and complementary to the peptide GCN4, Y. As a result, when the two fusion proteins are brought into contact with each other, a non-covalent binding interaction between the scFv and GCN4 peptide occurs, thereby physically retaining the two fusion proteins in the form of a complex mimicking an antibody comprising two antigen-binding portions on the same molecule (FIG. 1).

Purified Fab-X and Fab-Y with varying specificities were incubated together for 60 minutes (in a 37° C./5% CO₂ environment) at an equimolar concentration. The final molarity of each tested complex was 100 nM. In 384-well tissue culture plates, 1.0×10⁵ PBMC were added to wells, to which were added pre-formed Fab-X/Fab-Y bispecific antibodies. Following addition of the bispecific antibodies, the cells were incubated for 48 hours at 37° C./5% CO₂, with 250 ng/mL (final concentration) anti-human CD3 antibody (clone UCHT-1). After 48 hours the plates were centrifuged at 500×g for 5 minutes at 4° C. Cell culture conditioned media was transferred from the cell pellets to fresh plates and frozen at −80° C. Cells were washed with 60 μL FACS buffer two times by centrifugation, followed by resuspension of the pellets by shaking the plates at 2200 rpm for 30 seconds. The cells were stained with a cocktail of fluorescently labelled antibodies as listed in the Table 2 and incubated at 4° C. in the dark for 1 hour.

TABLE 2

| Epitope | Fluorophore | Clone | Source | Dilution |
|---|---|---|---|---|
| CD56 | APC | HCD56 | BioLegend ® | 40 |
| CD14 | PE/Cy7 | M5E2 | BD Pharmagen ® | 80 |
| CD8 | BV650 | RPA-T8 | BioLegend ® | 80 |
| CD4 | BV605 | RPA-T4 | BD Horizon ® | 40 |
| CD19 | PE-Cy5 | HIB19 | BD Pharmagen ® | 20 |
| CD45RO | BV786 | UCHL1 | BD Horizon ® | 40 |
| CD71 | FITC | M-A712 | BD Pharmagen ® | 20 |
| CD25 | BV510 | M-A251 | BD Horizon ® | 40 |
| CD137 | PE | 4B4-1 | BD Pharmagen ® | 20 |
| Live/Dead | Near IR | | Thermo Scientific ® | 1000 |

After this time, cells were washed twice with FACS buffer, before fixing with 2% paraformaldehyde (BD CellFix™, diluted in dH₂O) and stored overnight at 4° C. The plates were centrifuged at 500×g for 5 minutes, the fixation buffer aspirated to waste and the cells resuspended in a residual volume of 10 μL for acquisition on the iQue® Screener Plus (IntelliCyt®). The data analysis software package ForeCyt™ (IntelliCyt®) was used to gate on CD14+ monocytes, CD56+ NK cells, CD19+ B-cells, CD4+ and CD8+ memory and naïve T-cells. For each cell population the cellular expression of CD25, CD71 and CD137 was measured as reported median fluorescent intensity (MFI) values. The data were then used to calculate the log 2 fold changes of expression relative to control well values.

The resulting flow cytometry data were analysed by identification of the CD4 and CD8 memory and naïve T cells and well as the B cells, NK cells and monocytes. The MFI of the 3 activation markers CD71, CD25 and CD137 were then determined and the Log 2 fold changes calculated relative to the control wells for each cell population. These results were deposited into the visualisation software, Spotfire®, where it was possible to identify antigen pairs capable of inducing an increase in T cell activation.

When considering antigen pairs capable of upregulating the later activation markers CD25, CD71 and CD137 on T cells in the presence of an anti-CD3 stimulation, but not in unstimulated conditions, CD137-CD9 pair was identified.

The CD137-CD9 bispecific antibody was therefore taken into subsequent assays to show that its effect was repeatable across a larger number of donors.

### Example 2: CD137-CD9 Follow Up Assay

To confirm the effect of an anti-CD137-CD9 antibody on stimulation of T cells, a further 4 PBMC donors were assayed in stimulated and unstimulated conditions.

A grid of fusion proteins Fab-X and Fab-Y were created by diluting equimolar (1 μM) quantities of Fab-X (Fab-scFv) and Fab-Y (Fab-peptide) with specificity for CD9 and CD137 in TexMACS™ media (Miltenyi Biotec®) containing 100 U/mL penicillin/100 μg/mL streptomycin. Mixtures of equimolar (1 μM) Fab-Y proteins were also generated in the same manner. The Fab-X and Fab-Y fusion proteins were incubated together for 1 hour (in a 37° C./5% CO₂ environment), at a final concentration of 500 nM. Negative control wells contained TexMACS™ media only were also generated alongside the Fab-X and Fab-Y wells.

During this time, cryopreserved human PBMCs isolated from platelet leukapheresis cones were thawed and washed in TexMACS™ media and resuspended at 3.33×10⁶ cells/mL. The PBMCs were then seeded into 384 well flat bottom tissue culture plates (Greiner Bio-one®) at 30 μl/well (1×10⁵ PBMCs). A total of 10 μl of Fab-X/Fab-Y bispecific antibodies were transferred to the plates containing 30 μl PBMCs. The PBMCs were then either left unstimulated by the addition of 10 μl of TexMACS™ media, or stimulated with 10 μl of either soluble anti-CD3 (clone UCHT-1) (250 or 10 ng/mL final concentration) or soluble antigen staphylococcal enterotoxin B; SEB (1 μg/mL final concentration). This resulted in a final assay concentration of Fab-X/Fab-Y bispecific antibodies of 100 nM. The plates were then returned to a 37° C./5%002 environment for 48 hours.

After 48 hours the plates were centrifuged at 500×g for 5 minutes at 4° C. Cell culture conditioned media was transferred from the cell pellets to fresh plates and frozen at ⁻80° C. Cells were washed with 60 μL FACS buffer two times by centrifugation, followed by resuspension of the pellets by shaking the plates at 2200 rpm for 30 seconds. The cells were stained with a cocktail of fluorescently labelled antibodies as listed in Table 1 and incubated at 4° C. in the dark for 1 hour. After this time, cells were washed twice with FACS buffer, before fixing with 2% paraformaldehyde (BD CellFix™, diluted in dH₂O) and stored overnight at 4° C. The plates were centrifuged at 500×g for 5 minutes, the fixation buffer aspirated to waste and the cells resuspended in a residual volume of 10 μL for acquisition on the iQue® Screener Plus (IntelliCyt®).

The data analysis software package ForeCyt™ (IntelliCyt®) was used to gate on CD14+ monocytes, CD56+NK cells, CD19+ B-cells, CD4+ and CD8+ memory and naïve T-cells. For each cell population the cellular expression of CD25, CD71 and CD137 was measured as reported median fluorescent intensity values. The data were then used to calculate the log 2 fold changes of expression relative to control well values.

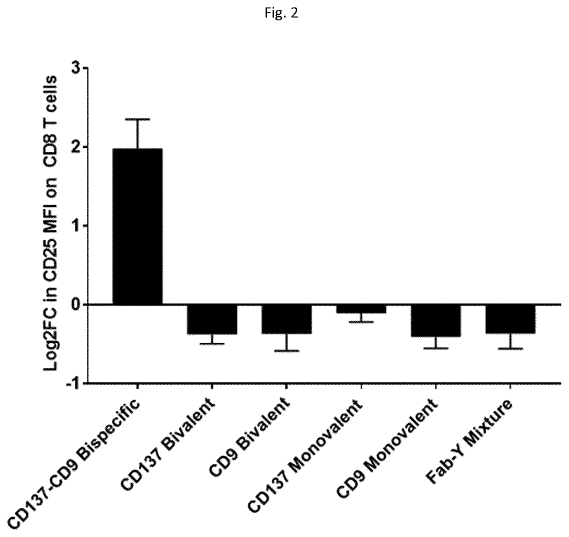

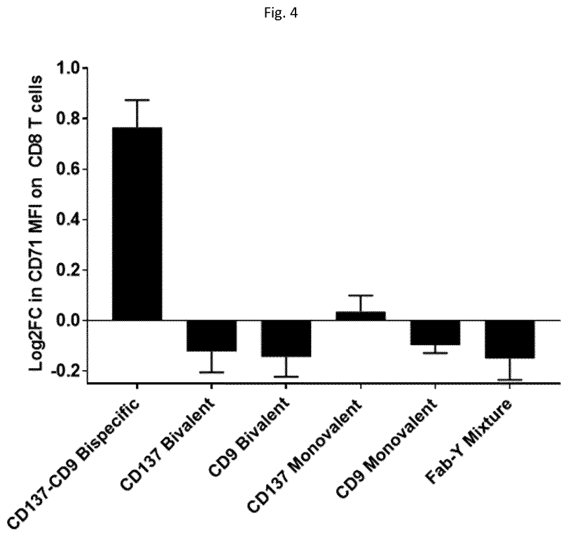

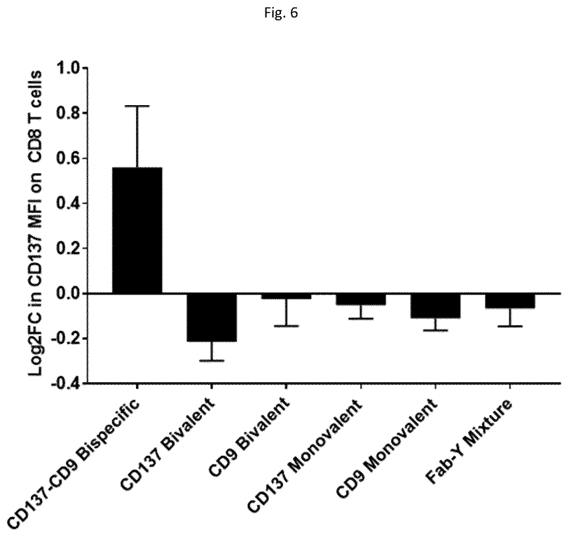

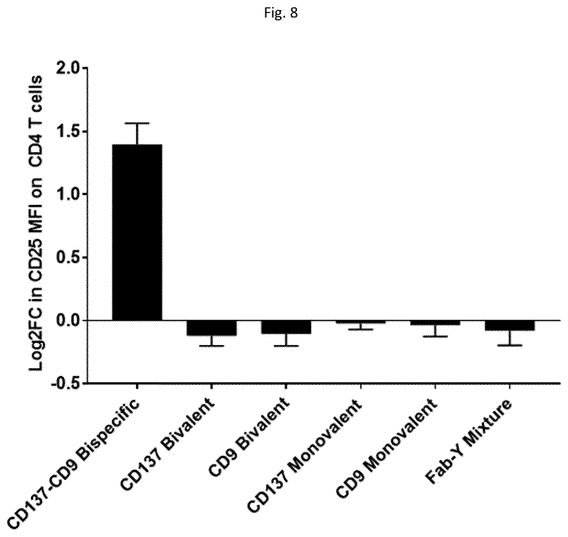

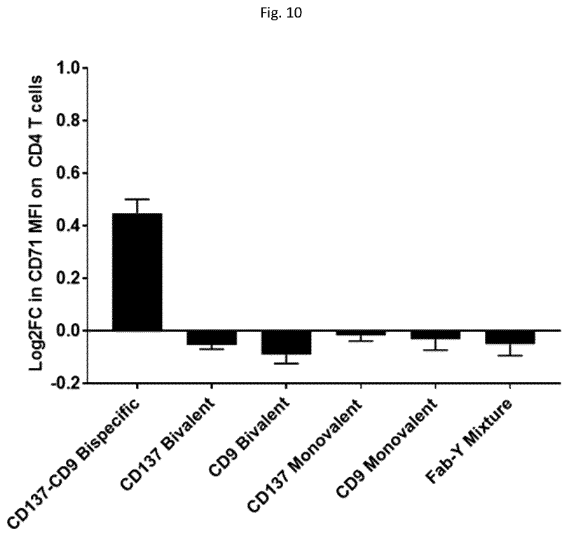

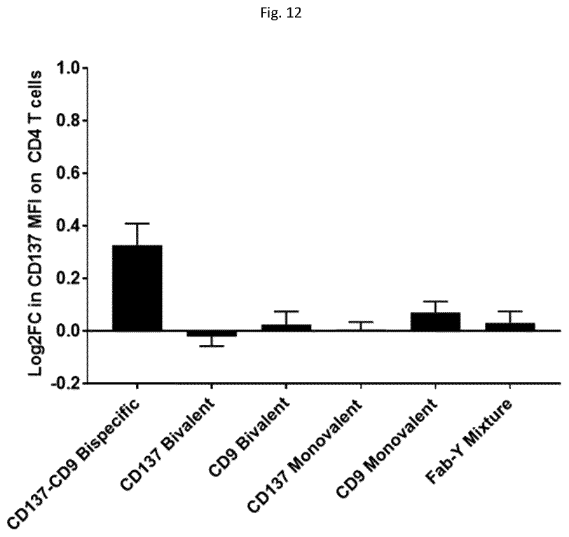

Seven Fab-X/Fab-Y bispecific antibodies showed increased expression of activation markers CD71, CD25 and CD137 on CD4+ and CD8+ T cells with anti-CD3 stimulation. FIGS. 2 to 13 show an example bispecific combination as well as the bivalent, monovalent and Fab-Y mixture controls. The CD137/CD9 bispecific antibody is shown to increase the expression of all three activation markers on CD8+ cells (FIG. 2: CD25; FIG. 4: CD71; FIG. 6: CD137) and on CD4+ cells (FIG. 8: CD25; FIG. 10: CD71; FIG. 12: CD137) when added to PBMCs stimulated with soluble anti-CD3 for 48 hours. The bivalents antibodies, formed by fusions where both Fab in the Fab-X/Fab-Y antibody are specific for either CD137 or CD9 (as the case may be) and monovalent antibodies for CD137 and CD9, formed by fusions where the Fab in the Fab-X is specific for CD137 or CD9 but the Fab in the Fab-Y is a negative control (in the present case the Fab of an anti-idiotypic antibody, hence not for an antigen expressed on or by any cell) did not lead to a similar increase in the activation marker fluorescence intensity on either cell populations. This confirms that neither the binding of CD137 alone or CD9 alone can stimulate activation in the absence of the other. Furthermore, the mixture of Fab-Y binding CD137 and Fab-Y binding CD9 also had no stimulatory effect on the T cell populations. This further confirms that there is a requirement, for the stimulatory function to occur, for the antigen-binding portions binding CD137 and CD9 to be on the same molecule, or on separate molecules which become associated via non-covalent or covalent associations or linkers.

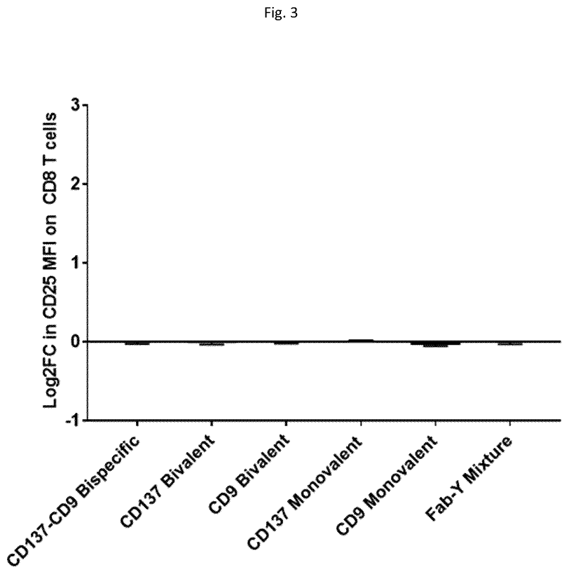

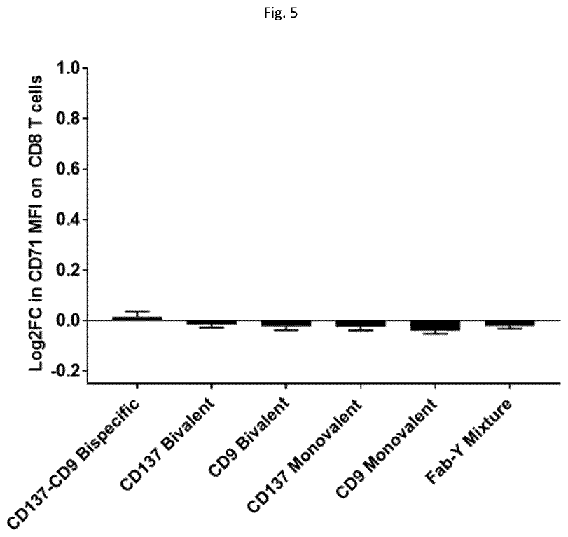

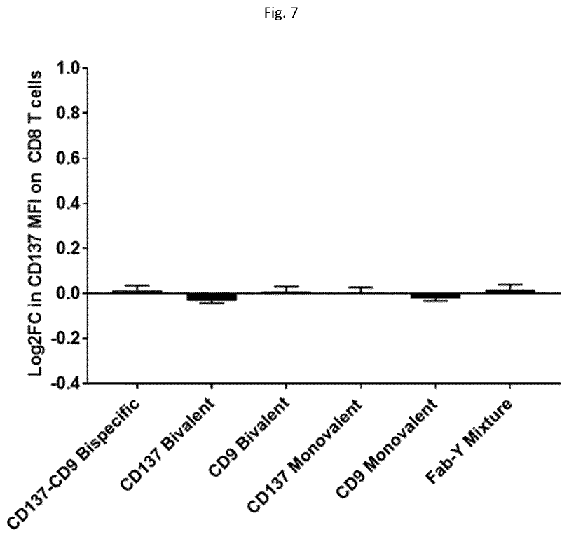

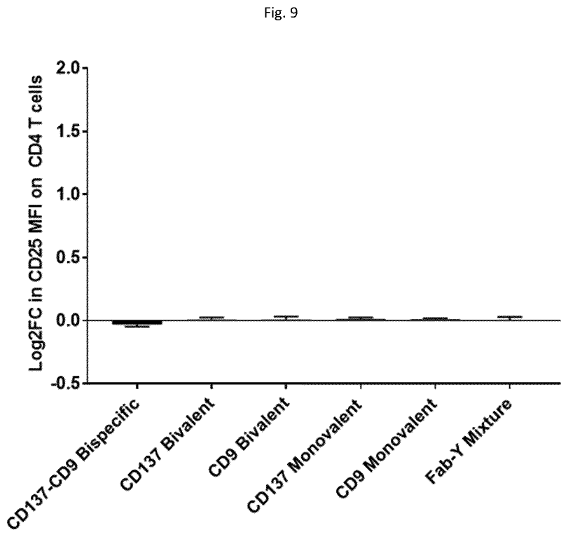

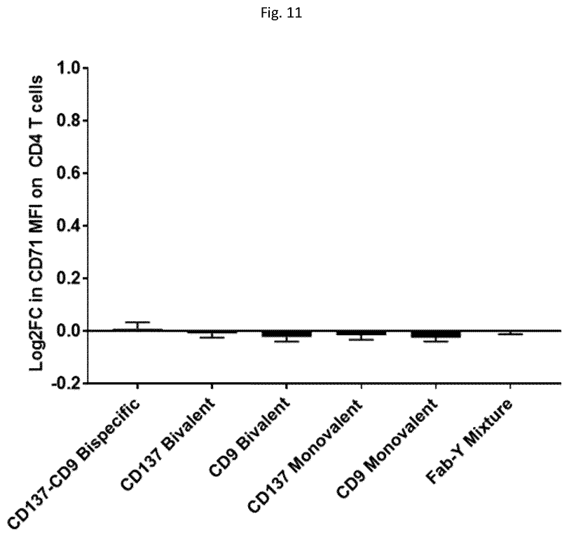

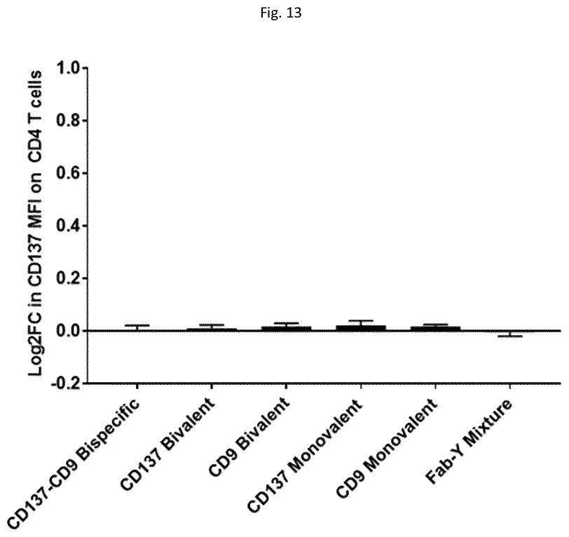

When studied in unstimulated conditions the CD137-CD9 bispecific antibodies did not lead to any increases in activation marker fluorescence intensity in either CD8+ cells (FIG. 3: CD25; FIG. 5: CD71; FIG. 7: CD137) or CD4+ cells (FIG. 9: CD25; FIG. 11: CD71; FIG. 13: CD137). This confirms that the binding of both CD137 and CD9 does not lead to the unwanted activation of resting T cells.

The statistical significance of the log 2 fold changes in activation markers of the CD137-CD9 antibodies, relative to the bivalent, monovalent and mixture controls, in stimulated or unstimulated conditions, is shown in Table 3.

TABLE 3

| | Activation marker | | |
|---|---|---|---|
| | CD25 | CD71 | CD137 |
| CD8 T cells in anti-CD3 | | | |
| CD137-CD9 Bispecific vs. CD137 Bivalent | <0.0001 | <0.0001 | 0.001 |
| CD137-CD9 Bispecific vs. CD9 Bivalent | <0.0001 | <0.0001 | 0.018 |
| CD137-CD9 Bispecific vs. CD137 | <0.0001 | <0.0001 | 0.0124 |
| Monovalent | | | |
| CD137-CD9 Bispecific vs. CD9 Monovalent | <0.0001 | <0.0001 | 0.0051 |
| CD137-CD9 Bispecific vs. Fab-Y Mixture | <0.0001 | <0.0001 | 0.0099 |
| CD8 T cells in Unstimulated | | | |
| CD137-CD9 Bispecific vs. CD137 Bivalent | 0.9972 | 0.6715 | 0.4274 |
| CD137-CD9 Bispecific vs. CD9 Bivalent | >0.9999 | 0.4582 | >0.9999 |
| CD137-CD9 Bispecific vs. CD137 | 0.9988 | 0.3708 | 0.9997 |
| Monovalent | | | |
| CD137-CD9 Bispecific vs. CD9 Monovalent | 0.6073 | 0.1 | 0.7016 |
| CD137-CD9 Bispecific vs. Fab-Y Mixture | >0.9999 | 0.4876 | >0.9999 |
| CD4 T cells in anti-CD3 | | | |
| CD137-CD9 Bispecific vs. CD137 Bivalent | <0.0001 | <0.0001 | <0.0001 |
| CD137-CD9 Bispecific vs. CD9 Bivalent | <0.0001 | <0.0001 | 0.0005 |
| CD137-CD9 Bispecific vs. CD137 | <0.0001 | <0.0001 | 0.0002 |
| Monovalent | | | |
| CD137-CD9 Bispecific vs. CD9 Monovalent | <0.0001 | <0.0001 | 0.0036 |
| CD137-CD9 Bispecific vs. Fab-Y Mixture | <0.0001 | <0.0001 | 0.0006 |
| CD4T cells in Unstimulated | | | |
| CD137-CD9 Bispecific vs. CD137 Bivalent | 0.5777 | 0.975 | 0.9998 |
| CD137-CD9 Bispecific vs. CD9 Bivalent | 0.5576 | 0.7361 | 0.9816 |
| CD137-CD9 Bispecific vs. CD137 | 0.4615 | 0.8693 | 0.9424 |
| Monovalent | | | |
| CD137-CD9 Bispecific vs. CD9 Monovalent | 0.5395 | 0.6266 | 0.9812 |
| CD137-CD9 Bispecific vs. Fab-Y Mixture | 0.7487 | 0.9966 | 0.9898 |

Statistical analysis was performed in GraphPad Prism®, and a One-way analysis of variance (ANOVA) was used with multiple comparisons (Sidak's multiple comparison test). Values presented represent the p values calculated from the Sidak's multiple comparison test, with p <0.01 taken to be statistically significant; n=4 donors, 2 technical replicates.

### Example 3: Comparison of Fab-X/Fab-Y Bispecific Antibodies with IgG Single Specificity Controls and Mixtures

To confirm the need for the antigen-binding portions binding CD137 and CD9 to be on the same antibody, in order to elicit the stimulatory effect, IgG1 antibodies were generated. These antibodies are monospecific and bivalent, which means they comprise each two antigen-binding portions for the same antigen, either CD137 or CD9. The IgG antibodies were then assayed individually and as a mixture on 4 PBMC donors in anti-CD3 conditions and compared to the corresponding Fab-X/Fab-Y bispecific antibodies and controls.

A grid of Fab-X and Fab-Y fusion proteins were created by combining equimolar (1 μM) quantities of Fab-X (Fab-scFv) and Fab-Y (Fab-peptide) with specificity for CD9 and CD137 in TexMACS™ media (Miltenyi Biotec®) containing 100 U/mL penicillin/100 μg/mL streptomycin. Mixtures of equimolar (1 μM) Fab-Y proteins were also generated in the same manner. The IgG antibodies were diluted to 500 nM individually or as a mixture. The Fab-X and Fab-Y fusion proteins were incubated together for 1 hour (in a 37° C./5% CO₂ environment), at a final concentration of 500 nM. Negative control wells contained TexMACS™ media only were also generated alongside the Fab-X and Fab-Y wells.

During this time, cryopreserved human PBMCs isolated from platelet leukapheresis cones were thawed and washed in TexMACS™ media and resuspended at 3.33×10⁶ cells/mL. The PBMCs were then seeded into 384 well flat bottom tissue culture plates (Greiner Bio-one®) at 30 μl/well (1×10⁵ PBMCs).

A total of 10 μl of Fab-X/Fab-Y bispecific antibodies or IgG antibodies were transferred to the plates containing 30 μl PBMCs. The PBMCs were then either left unstimulated by the addition of 10 μl of TexMACS™ media, or stimulated with 10 μl soluble anti-CD3 (clone UCHT-1) (10 ng/mL final concentration). This resulted in a final assay concentration of Fab-X/Fab-Y bispecific antibodies of 100 nM. The plates were then returned to a 37° C./5% CO₂ environment for 48 hours.

After 48 hours the plates were centrifuged at 500×g for 5 minutes at 4° C. Cell culture conditioned media was transferred from the cell pellets to fresh plates and frozen at ⁻80° C. Cells were washed with 60 μL FACS buffer two times by centrifugation, followed by resuspension of the pellets by shaking the plates at 2200 rpm for 30 seconds. The cells were stained with a cocktail of fluorescently labelled antibodies as listed in Table 1 and incubated at 4° C. in the dark for 1 hour. After this time, cells were washed twice with FACS buffer, before fixing with 2% paraformaldehyde (BD CellFix™, diluted in dH₂O) and stored overnight at 4° C. The plates were centrifuged at 500×g for 5 minutes, the fixation buffer aspirated to waste and the cells resuspended in a residual volume of 10 μL for acquisition on the iQue® Screener Plus (IntelliCyt®). The data analysis software package ForeCyt™ (IntelliCyt®) was used to gate on CD14+ monocytes, CD56+NK cells, CD19+ B-cells, CD4+ and CD8+ memory and naïve T-cells. For each cell population the cellular expression of CD25 and CD71 was measured as reported median fluorescent intensity values. The data were then used to calculate the log 2 fold changes of expression relative to control well values.

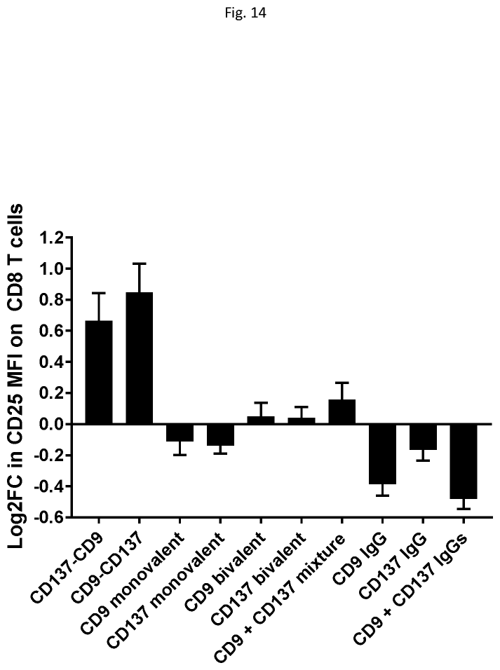

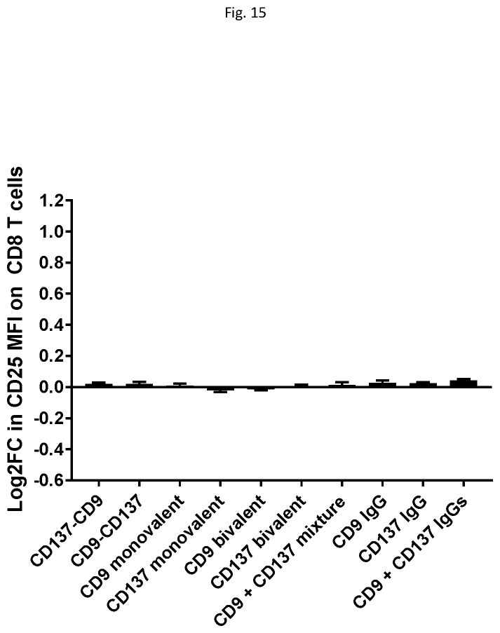

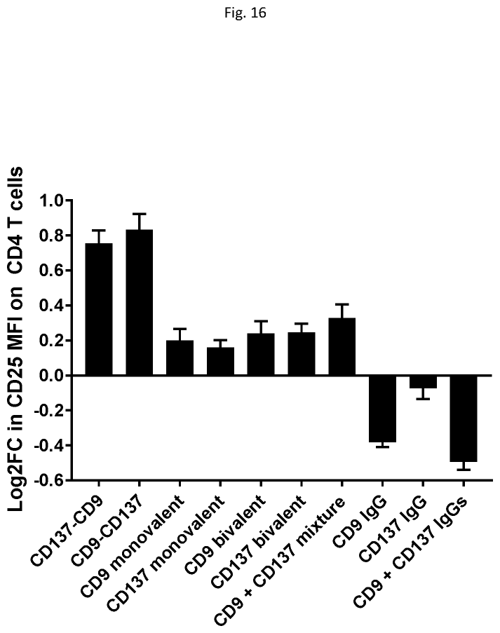

As previously shown the CD137-CD9 bispecific antibodies caused an increase in the level of CD25 on both CD4+ and CD8+ T cells when anti-CD3 stimulated conditions (FIGS. 14 and 16, first and second bars). In contrast, the monovalent and bivalent antibodies caused no change (FIGS. 14 and 16), irrespective on the orientation of the antigen-binding portions within the antibody. The anti-CD9 and anti-CD137 IgG antibodies, either individually or as a mixture, also did not lead to an increase in CD25 or CD71 expression on the T cell populations (FIGS. 1,16, 18 and 20, last three bars). No effect could be seen by any antibody treatment in unstimulated conditions (FIGS. 15,17,19 and 21).

### Example 4: Effect of CD137-CD9 Bispecific on T Cell Proliferation

The effect of an anti-CD137-CD9 antibody on proliferation of CD4+ and CD8+ T cells was assessed in 4 PBMC donors. Mixtures of fusion proteins Fab-X and Fab-Y were created by diluting equimolar (1 μM) quantities of Fab-X (Fab-scFv) and Fab-Y (Fab-peptide) with specificity for CD9 and CD137 in Dulbecco's Minimum Essential Medium (DMEM; ThermoFisher) containing 10% foetal bovine serum (FBS) and 100 U/mL penicillin/100 μg/mL streptomycin. Mixtures of equimolar (1 μM) Fab-Y proteins were also generated in the same manner. The Fab-X and Fab-Y fusion proteins were incubated together for 1 hour (in a 37° C./5% CO₂ environment).

During this time, cryopreserved human PBMCs isolated from platelet leukapheresis cones were thawed, washed in DMEM media and resuspended at approximately 2×10⁶ cells/mL in PBS. Cell Trace™ Violet (CTV; ThermoFisher) was then added to a final concentration of 10 μM (2 μl 5 mM CTV in DMSO added to 10 mL cells), incubated in the dark at room temperature for 10 minutes, the cells washed twice with DMEM and resuspended in media to a final concentration of 1×10⁶ cells/mL.

Fab-X/Fab-Y bispecific antibodies were diluted to 400 nM concentration in DMEM and 50 μl transferred in triplicate to wells of a 96 well U bottom tissue culture plate. Anti-CD3 (clone UCHT-1), 50 μl of a 40 ng/mL solution in DMEM, was then added. To 3 wells 100 μl DMEM media alone was added as a negative proliferation control. Finally, 100 μl of CTV labelled PBMC were added to each well. This resulted in a final assay concentration of Fab-X/Fab-Y bispecific antibodies of 100 nM, 10 ng/mL anti-CD3 and 1×10⁵ cells/well. The plates were then returned to a 37° C./5% CO₂ environment for 96 hours.

After 96 hours the plates were centrifuged at 500×g for 5 minutes. Cell culture conditioned media was removed and the cells resuspended in 100 μl PBS containing fluorescently labelled antibodies as listed in Table 4. The cells were incubated at room temperature in the dark for 15 minutes, washed with PBS, resuspended in 100 μl/per well PBS and analysed by flow cytometry (BD FACS Canto II™). Total events from 50 μl (50% volume) of each well were collected.

The data analysis software package FlowJo® was used to gate on CD8+ and CD4+ cells. Cell proliferation was assessed by enumerating cells with reduced CTV staining relative to unstimulated cells. The data are presented as the mean±SEM for triplicate wells.

TABLE 4

| Epitope | Fluorophore | Clone | Source | Dilution |
|---|---|---|---|---|
| CD8 | Pacific blue | HIT8a | BioLegend ® | 200 |
| CD4 | APC | RPA-T4 | BioLegend ® | 20 |

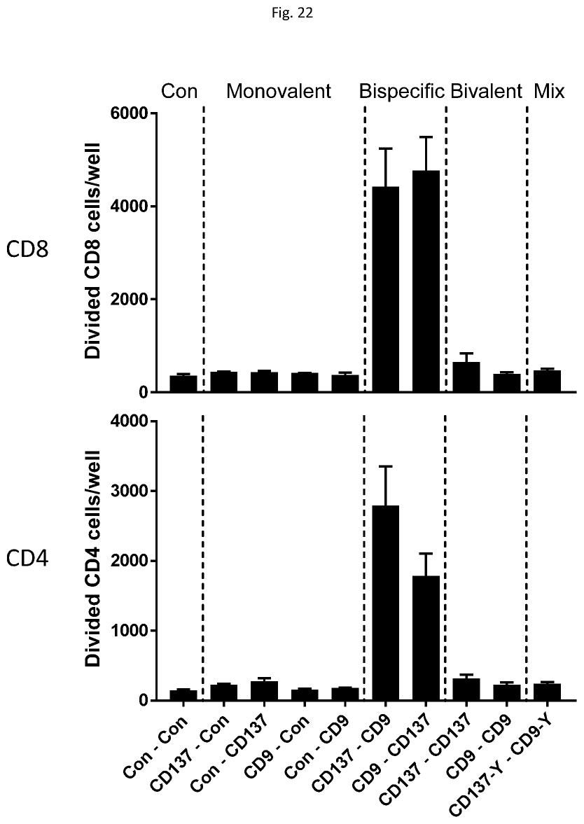

The bispecific anti-CD137/CD9 antibodies and anti-CD9/CD137 antibody are shown to increase the proliferation of both CD4+ and CD8+ T cells when added to PBMCs stimulated with soluble anti-CD3 for 96 hours. The bivalents antibodies formed by Fab-X and Fab-Y antibodies where both Fabs are specific for either CD137 or CD9 (as the case may be) and monovalent antibodies for CD137 and CD9, formed by fusions where the Fab in the Fab-X is specific for CD137 or CD9 but the Fab in the Fab-Y is a negative control (in the present case the Fab of an anti-idiotypic antibody, hence not for an antigen expressed on or by any cell) did not lead to a similar increase in proliferation in the absence of the other. Furthermore, the mixture of Fab-Y binding CD137 and Fab-Y binding CD9 also had no stimulatory effect on the T cell populations. This further confirms that there is a requirement, for the stimulatory function to occur, for the antigen-binding portions binding CD137 and CD9 to be on the same molecule, or on separate molecules which become associated via non-covalent or covalent associations or linkers (FIG. 22).

### Example 5: Antibody Concentration-Response Studies

The CD137-CD9 bispecific antibody was titrated in a proliferation assay using PBMC from 2 donors.

Mixtures of fusion proteins Fab-X and Fab-Y were created by diluting equimolar (1 μM) quantities of Fab-X (Fab-scFv) and Fab-Y (Fab-peptide) with specificity for CD9, CD137 or non-binding control as indicated in DMEM (ThermoFisher) containing 10% FBS and 100 U/mL penicillin/100 μg/mL streptomycin, then incubated together for 1 hour (in a 37° C./5% CO2 environment).

During this time, cryopreserved human PBMCs isolated from platelet leukapheresis cones were thawed, washed in DMEM media and resuspended at approx. 2×10⁶ cells/mL in PBS. Cell Trace™ Violet (CTV; ThermoFisher) was then added to a final concentration of 10 (2 μl 5 mM CTV in DMSO added to 10 mL cells), incubated in the dark at room temperature for 10 minutes, the cells washed twice with DMEM and resuspended in media to a final concentration of 1×10⁶ cells/mL.

Fab-X/Fab-Y bispecific antibodies were diluted to 400 nM concentration in DMEM then 1:10 and 1:100 dilutions of this made also in DMEM. Aliquots of 50 μl were then transferred in triplicate to wells of a 96 well U bottom tissue culture plate. Anti-CD3 (clone UCHT-1), 50 μl of a 200 ng/mL solution in DMEM, was then added. To 3 wells 100 μl DMEM media alone was added as a negative proliferation control. Finally, 100 μl of CTV labelled PBMC was added to each well. This resulted in a final assay concentration of Fab-X and Fab-Y complexes of 100 nM, 10 nM or 1 nM plus 10 ng/mL anti-CD3 and 1×10⁵ cells/well. The plates were then returned to a 37° C./5% CO₂ environment for 96 hours.

After 96 hours the plates were centrifuged at 500×g for 5 minutes. Cell culture conditioned media was removed and the cells resuspended in 100 μl PBS containing fluorescently labelled antibodies as listed in Table 3 above. The cells were incubated at room temperature in the dark for 15 minutes, washed with PBS, resuspended in 100 μl/per well PBS and analysed by flow cytometry (BD FACS Canto II™). Total events from 50 μl (50% volume) of each well were collected.

The data analysis software package FlowJo® was used to gate on CD4+ and CD8+ cells. Cell proliferation was assessed by enumerating cells with reduced CTV staining relative to unstimulated cells. The data are presented as the mean±SEM for triplicate wells.

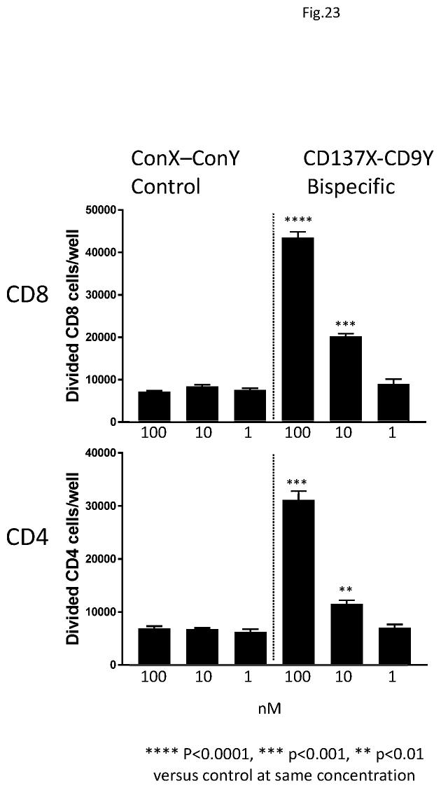

As shown in FIG. 23, an anti-CD137/CD9 bispecific antibody was capable of enhancing T-cell proliferation and this effect was concentration-dependent. Statistically significant differences are highlighted with p values <0.01 using unpaired t test (Graphpad Prism®).

### Example 6: Comparison of Fab-X/Fab-Y Bispecific Antibodies with Bispecific IgGs

To investigate whether the Fab-X/Fab-Y bispecific antibodies against CD137 and CD9 would achieve the same effect when converted into an IgG format, the variable regions of the Fab-X and Fab-Y fusion proteins were cloned into a bispecific IgG format using established methods available in the public domain. Bivalent monospecific IgG against either CD9 or CD137 were also generated. The functionality of targeting CD9 and CD137 in the different bispecific formats was tested by comparing them in an anti-CD3 (clone UCHT-1) stimulated PBMC assay.

Fab-X/Fab-Y bispecific antibodies were created by adding equimolar quantities of Fab-X (Fab-scFv) and Fab-Y (Fab-peptide) with specificity for CD9 and CD137 in TexMACS™ media (Miltenyi Biotec®) containing 100 U/mL penicillin/100 μg/mL streptomycin. The Fab-X and Fab-Y fusion proteins were incubated together for 1 hour (in a 37° C., 5% CO₂ environment), at a final concentration of 2 μM. Full-length IgG constructs were diluted to 2 μM in TexMACS™ media containing 100 U/mL penicillin/100 μg/mL streptomycin. Mixtures of the bivalent monospecific IgG controls were also generated at 2 μM. After incubation, the antibodies were diluted to 500 nM in TexMACS™ media. Negative control wells containing TexMACS™ media only were also generated alongside.

During the incubation, cryopreserved human PBMC isolated from leukoreduction system chambers were thawed and washed in TexMACS™ media and resuspended at 2.5×10⁶ cells/mL. The PBMC were then seeded into 96-well U-bottom tissue culture plates)(Costar® at 60 μL/well (1.5×10⁵ PBMC). A total of 20 μL of Fab-X/Fab-Y bispecific antibodies or full-length IgG constructs were transferred to the plates containing 60 μL PBMC. The PBMC were then stimulated with 20 μL of soluble anti-CD3 (clone UCHT-1) at 10 ng/mL final concentration. This resulted in a final assay concentration of antibodies of 100 nM. Negative control wells had 20 μL of TexMACS™ media added instead of antibody. The plates were then returned to a 37° C., 5% CO₂ environment for 48 hours.

After 48 hours the plates were washed with 100 μL FACS buffer and centrifuged at 500×g for 5 minutes at 4° C. The cells were washed with 180 μL FACS buffer followed by centrifugation and by resuspension of the pellets by shaking the plates at 1800 rpm for 30 seconds. The cells were stained with a cocktail of fluorescently labelled antibodies as listed in Table 1 and incubated at 4° C. in the dark for 1 hour. After this time, cells were washed twice with FACS buffer, before fixing with 2% paraformaldehyde (BD CellFix™, diluted in dH₂O) and incubating for 1 hour at 4° C. 100 μL of FACS buffer was added to the plates, they were centrifuged at 500×g for 5 minutes, the fixation buffer aspirated to waste and the cells resuspended in a residual volume of 15 μL for acquisition on the iQue® Screener Plus (IntelliCyt®).

The data analysis software package ForeCyt™ (IntelliCyt®) was used to gate on CD14+ monocytes, CD56+NK cells, CD19+ B cells, CD4+ and CD8+ memory and naïve T cells. For each cell population the cellular expression of CD25 and CD71 was measured as reported median fluorescent intensity values. The data were then used to calculate the log 2 fold changes of expression relative to control well values.

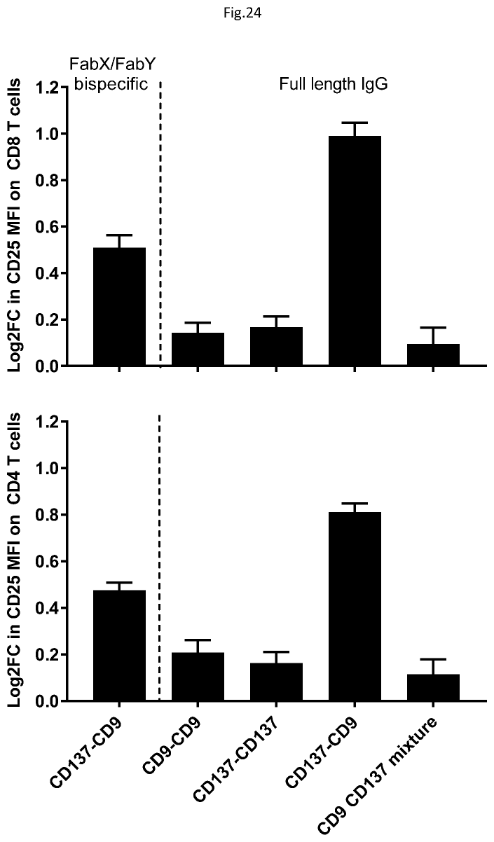

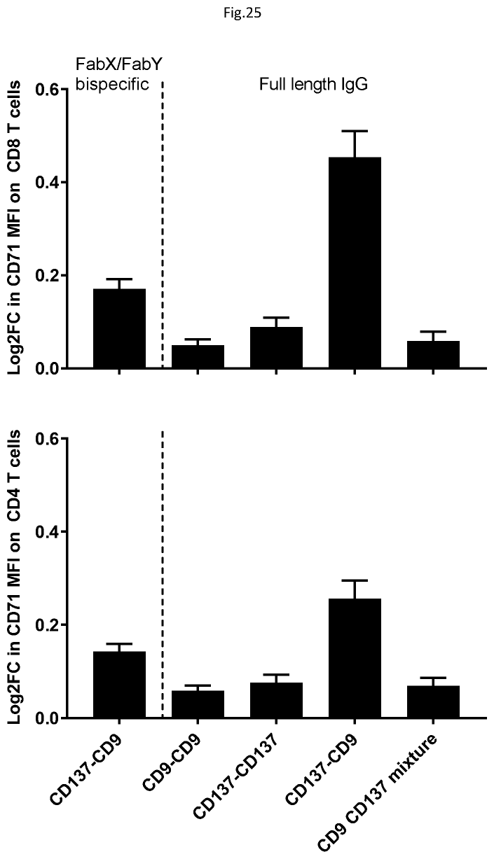

FIG. 24 (for CD25) and FIG. 25 (for CD71) show that the Fab-X/Fab-Y format and the IgG format exhibit very similar effects in increasing CD25 and CD71 expression in activated CD8+ and CD4+ T-cells, respectively. In contrast, the effect of monospecific bivalent anti-CD137 or anti-CD9 IgG antibodies or their mixture did not elicit any effect.

### Example 7: Concentration-Dependent Studies of Anti-CD137/CD9 Bispecific IgG

The IgG bispecific antibodies against CD137 and CD9 of example 6 were then titrated in an anti-CD3 (clone UCHT-1) stimulated PBMC assay, alongside each monospecific bivalent IgG antibodies against CD9 and CD137 or their mixture.

The IgG antibodies were diluted to 2 μM in TexMACS™ media containing 100 U/mL penicillin/100 μg/mL streptomycin. Mixtures of the bivalent IgG controls were also generated at 2 μM. After incubation, a 6-point 4-fold titration was generated in TexMACS™ media. Negative control wells containing TexMACS™ media only were also generated alongside.

During the incubation, cryopreserved human PBMC isolated from leukoreduction system chambers were thawed and washed in TexMACS™ media and resuspended at 2.5×10⁶ cells/mL. The PBMC were then seeded into 96-well U-bottom tissue culture plates (Costar®) at 60 μL/well (1.5×10⁵ PBMC). A total of 20 μL of full-length IgG constructs were transferred to the plates containing 60 μL PBMC. The PBMC were then stimulated with 20 μL of soluble anti-CD3 (clone UCHT-1) at 10 ng/mL final concentration. This resulted in a final assay concentration of antibodies of 400 nM top concentration. Negative control wells had 20 μL of TexMACS™ media added instead of antibody. The plates were then returned to a 37° C., 5% CO₂ environment for 48 hours.

After 48 hours the plates were washed with 100 μL FACS buffer and centrifuged at 500×g for 5 minutes at 4° C. The cells were washed with 180 μL FACS buffer followed by centrifugation and by resuspension of the pellets by shaking the plates at 1800 rpm for 30 seconds. The cells were stained with a cocktail of fluorescently labelled antibodies as listed in Table 2 and incubated at 4° C. in the dark for 1 hour. After this time, cells were washed twice with FACS buffer, before fixing with 2% paraformaldehyde (BD CellFix™, diluted in dH₂O) and incubating for 1 hour at 4° C. 100 μL of FACS buffer was added to the plates, they were centrifuged at 500×g for 5 minutes, the fixation buffer aspirated to waste and the cells resuspended in a residual volume of 15 μL for acquisition on the iQue® Screener Plus (IntelliCyt®).

The data analysis software package ForeCyt™ (IntelliCyt®) was used to gate on CD14÷ monocytes, CD56+NK cells, CD19+ B cells, CD4+ and CD8+ memory and naïve T cells. For each cell population the cellular expression of CD25 was measured as reported median fluorescent intensity values. The data were then used to calculate the log 2 fold changes of expression relative to control well values.

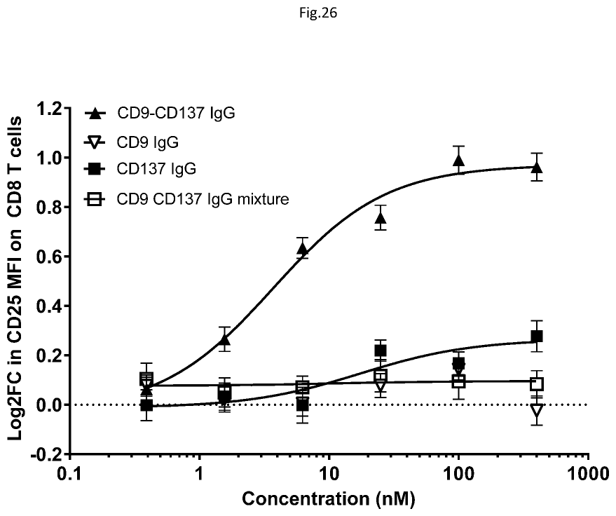

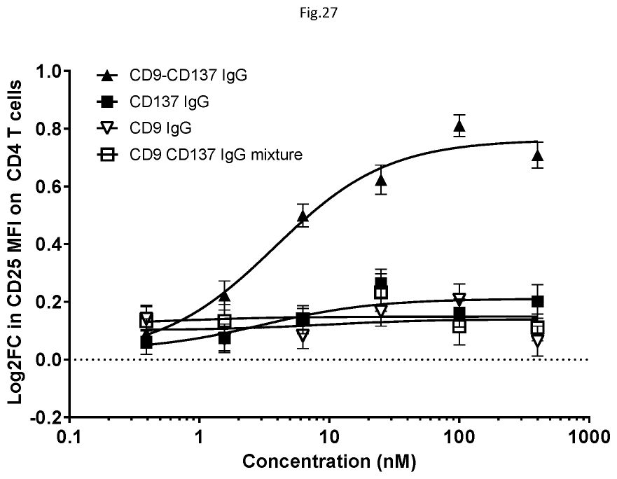

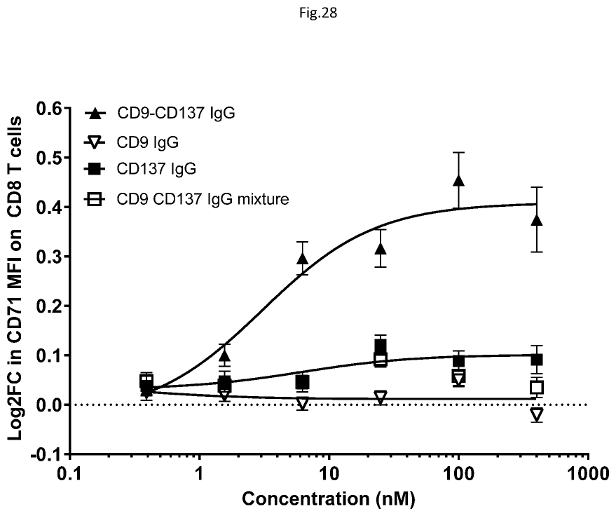

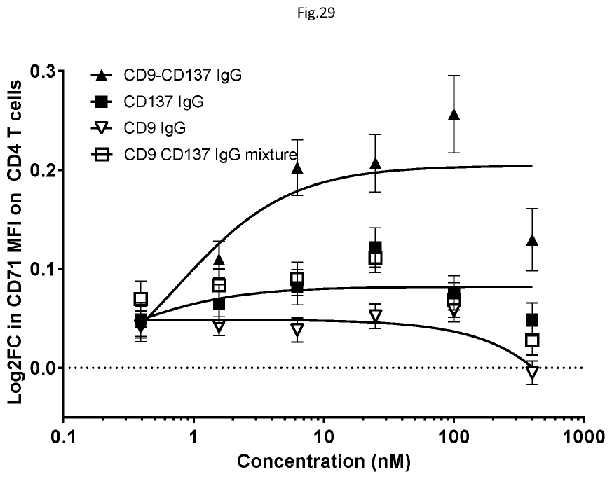

As shown in FIGS. 26-29, the IgG bispecific antibodies binding both CD137 and CD9 caused a concentration-dependent increase in the expression of CD25 and CD71 on activated CD8+ and CD4+ T cells in a concentration dependent fashion. In contrast, the monospecific bivalent antibodies against either CD137 or CD9, or their mixture did not show any effect.

### Example 8: Identification of Functional CD137 Variable Regions

To identify additional variable regions against CD137 which lead to the activation of T cells when complexed with CD9 antibodies, a further 3 PBMC donors were assayed in anti-CD3 stimulated conditions. These donors were treated with CD137-CD9 bispecific antibodies generated from nine CD137 variable regions and seven CD9 variable regions.

These different variable regions (with a numerical identifier) were combined with each other and a negative control V region (5599) to generate bispecific, bivalent and monovalent combinations in a grid format as represented in Table 5.

TABLE 5

| Fab-X | | | | | | | | | |
|---|---|---|---|---|---|---|---|---|---|
| CD9 | | | | | | | CD137 | | |
| 7270 | 7271 | 7272 | 7485 | 7486 | 7489 | 7491 | 8475 | 8476 | 8479 |

| | | | | |
|---|---|---|---|---|
| Fab-Y | CD9 | 7270 | Bivalent | Bispecific |
| | | 7271 | | |
| | | 7272 | | |
| | | 7485 | | |
| | | 7486 | | |
| | | 7489 | | |
| | | 7491 | | |
| | CD137 | 8475 | Bispecific | Bivalent |
| | | 7476 | | |
| | | 7479 | | |
| | | 11175 | | |
| | | 11176 | | |
| | | 11420 | | |

| | | |
|---|---|---|
| Con | 5599 | Monovalent |

| Fab-X | | | | | | |
|---|---|---|---|---|---|---|
| CD137 | | | | | | Con |
| 11175 | 11176 | 11420 | 11566 | 11568 | 11569 | 5599 |

| | | | | |
|---|---|---|---|---|
| Fab-Y | CD9 | 7270 | Bispecific | Monovalent |

7271
7272
7485
7486
7489
7491

| | | |
|---|---|---|
| CD137 | 8475 | Bivalent |

7476
7479
11175
11176
11420

| | | |
|---|---|---|
| Con | 5599 | Monovalent |

A grid of Fab-X and Fab-Y fusion proteins were created by diluting equimolar (1 μM) quantities of Fab-X (Fab-scFv) and Fab-Y (Fab-peptide) with specificity for CD9 and CD137 in TexMACS™ media (Miltenyi Biotec®) containing 100 U/mL penicillin/100 μg/mL streptomycin. The Fab-X and Fab-Y fusion proteins were incubated together for 1 hour (in a 37° C., 5% CO₂ environment), at a final concentration of 500 nM. Negative control wells containing TexMACS™ media only were also generated alongside the Fab-X and Fab-Y wells.

During this time, cryopreserved human PBMC isolated from platelet leukapheresis cones were thawed and washed in TexMACS™ media and resuspended at 2.5×10⁶ cells/mL. The PBMC were then seeded into 96-well U-bottom tissue culture plates (Costar®) at 60 μl/well (1.5×10⁵ PBMC). A total of 20 μl of Fab-X/Fab-Y bispecific antibodies were transferred to the plates containing 60 μL PBMC. The PBMC were then either left unstimulated by the addition of 20 μl of TexMACS™ media or stimulated with 20 μl of soluble anti-CD3 (clone UCHT-1) (10 ng/mL final concentration). This resulted in a final assay concentration of Fab-X/Fab-Y bispecific antibodies of 100 nM. The plates were then returned to a 37° C., 5% CO₂ environment for 48 hours.

After 48 hours the plates were centrifuged at 500×g for 5 minutes at 4° C. Cell culture conditioned media was transferred from the cell pellets to fresh plates and frozen at ⁻80° C. Cells were washed with 60 μl FACS buffer two times by centrifugation, followed by resuspension of the pellets by shaking the plates at 1800 rpm for 30 seconds. The cells were stained with a cocktail of fluorescently labelled antibodies as listed in Table 6 and incubated at 4° C. in the dark for 1 hour. After this time, cells were washed twice with FACS buffer, and fixed with 2% paraformaldehyde for one hour at 4° C. before adding 150 μl FACS buffer. The plates were centrifuged at 500×g for 5 minutes, the fixation buffer aspirated to waste and the cells resuspended in a residual volume of 15 μL for acquisition on the iQue® Screener Plus (IntelliCyt®).

TABLE 6

| Epitope | Fluorophore | Clone | Source | Dilution |
|---|---|---|---|---|
| CD56 | APC | HCD56 | BioLegend ® | 40 |
| CD14 | PE/Cy7 | M5E2 | BD Pharmagen ® | 80 |
| CD8 | BV650 | RPA-T8 | BioLegend ® | 80 |
| CD4 | BV605 | RPA-T4 | BD Horizon ® | 40 |
| CD19 | PE-Cy5 | HIB19 | BD Pharmagen ® | 20 |
| CD45RO | BV786 | UCHL1 | BD Horizon ® | 40 |
| CD71 | FITC | M-A712 | BD Pharmagen ® | 20 |
| CD25 | BV510 | M-A251 | BD Horizon ® | 40 |
| CD137 | PE | 4B4-1 | BD Pharmagen ® | 20 |
| Live/Dead | Near IR | | Thermo Scientific ® | 1000 |

The data analysis software package ForeCyt™ (IntelliCyt®) was used to gate on CD14+ monocytes, CD56+NK cells, CD19+ B-cells, CD4+ and CD8+ memory and naïve T cells. For each cell population, the cellular expression of CD25, CD71 and CD137 was measured as reported median fluorescent intensity values. The data were then used to calculate the Log 2 fold changes of expression relative to control well values.

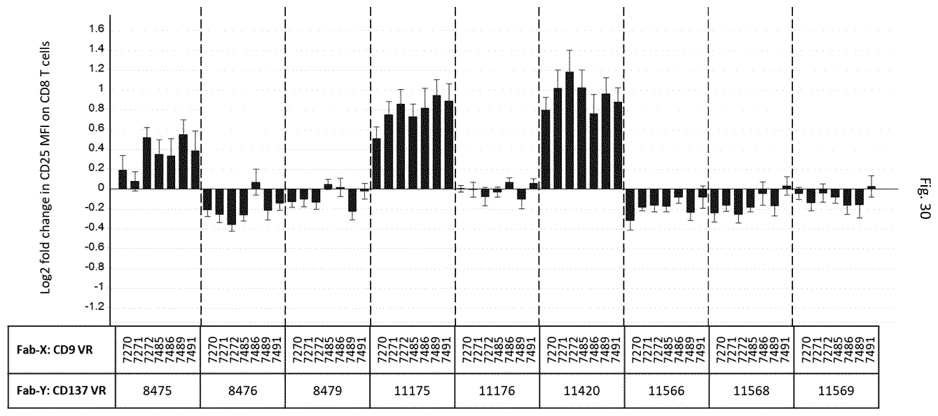

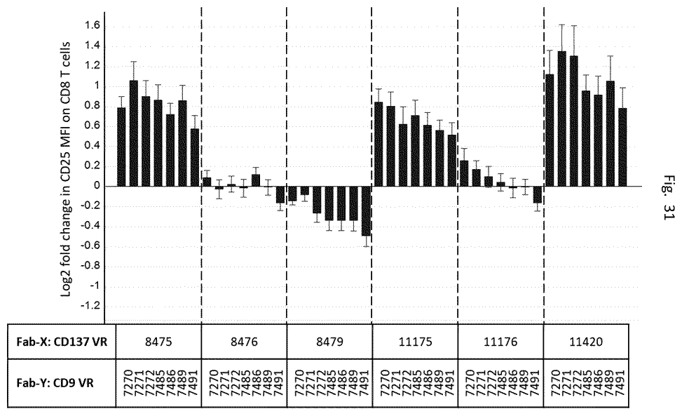

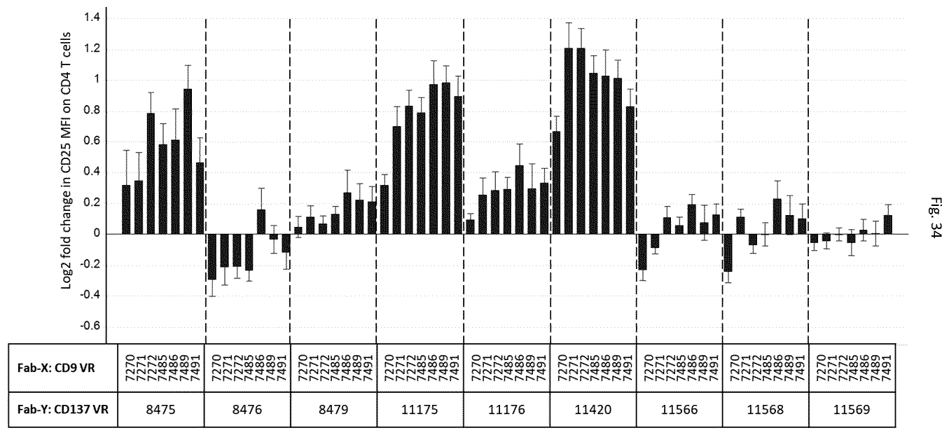

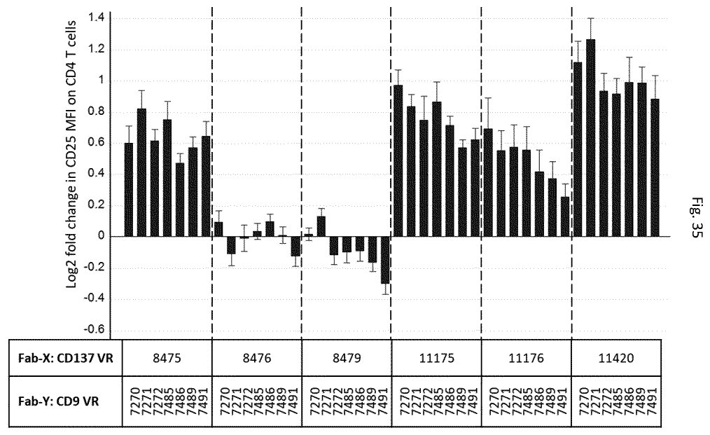

Three anti-CD137 variable regions (8475, 11175, 11420) led to increases of more than 0.5 Log 2 fold change in the level of CD25 expressed on CD8+ and CD4+ T cells when added as a bispecific with anti-CD9, irrespective of the orientation of the bispecific antibody (FIGS. 30, 31, 34, 35). A further variable region for CD137 (11176) increased CD25 on CD4+ T cells only. All 7 different anti-CD9 variable regions performed equally well.

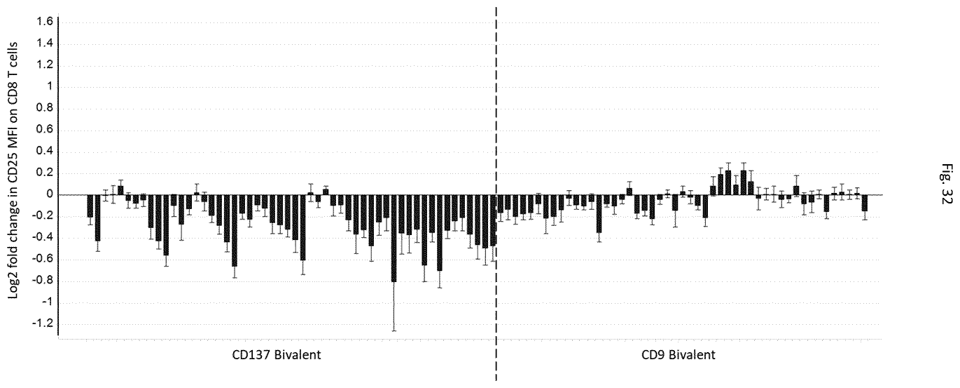

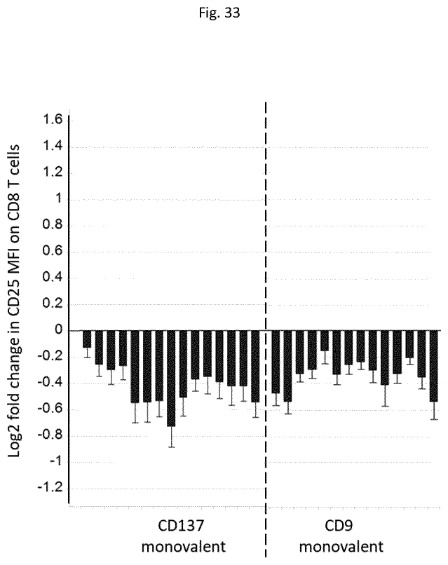

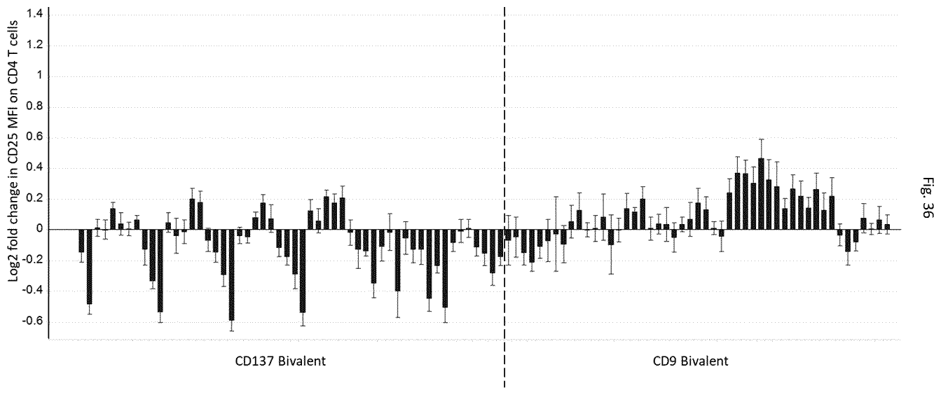

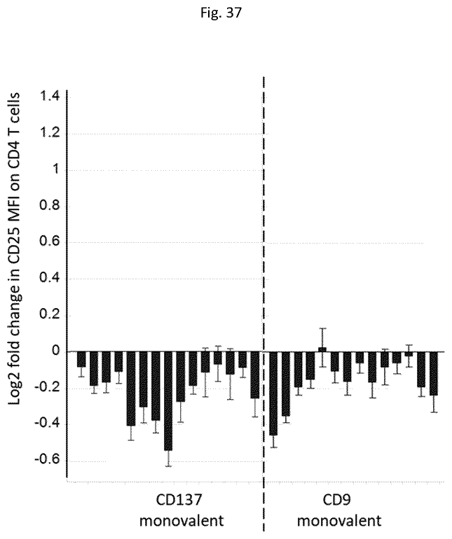

As expected, controls bivalent antibodies or monovalent Fab-X or Fab-Y antibodies made by the same variable regions against CD9 or CD137 did not lead to increases in CD25 levels (FIGS. 32, 33, 36, 37).

In order to assess which domain(s) of CD137 are responsible for such function, peptides sequences corresponding to different (or combinations of different) CD137 cysteine-rich domains (CRDs) were designed. These sequences contain the four CRDs present in CD137 and were designed to minimize changes in the three-dimensional structure of each domain: CRDs 2 and 3 were only analysed in combination with neighbouring domains and an even number of cysteine residues was present in each sequence.

DNA sequences coding for each peptide (as shown in Table 7) were ordered from Twist Bioscience in a mammalian expression vector with a human Fc tag at the C-terminus. Each plasmid was used to transfect Expi293F™ cells (Gibcon™) to generate the different peptide-human Fc fusion proteins. For transfections, 850 μl of Expi293F™ cells (3×10³ cells/mL) were plated in 48-well blocks and transfected with 3 μg of DNA using the ExpiFectamine™ 293 Transfection Kit (ThermoFisher) following the manufacturer's instructions. Cells were incubated in a 37° C., 5% CO₂ environment with shaking at 225 rpm. After 6 days, cells were pelleted (1000×g, 10 min) and the conditioned medias containing each of the peptides were harvested, quantitated and diluted to 1.7 μg/mL in Expi293™ Expression Medium (ThermoFisher). The conditioned medias were used for binding assessment to our panel of antibodies using the Octet® RED384 System (FortéBio) at room temperature with anti-hIgG Fc Capture (AHC) Biosensors (FortéBio). Firstly, an array of 8 sensors was dipped in kinetics buffer (PBS, 0.1% BSA, 0.02% Tween 20) for 180 seconds to provide a baseline signal. Sensors were then moved to wells with 200 μl of the conditioned medias containing each of the six different CD137 peptide-human Fc fusion proteins (diluted 1:5 in kinetics buffer), or kinetics buffer as a negative control, to immobilize the protein to the biosensor for 300 seconds, followed by a second baseline step (180 seconds) in kinetics buffer to equilibrate the biosensors now coated with the CD137 peptide-human Fc fusion proteins. No detachment of the peptides was observed during this step. Sensors were then dipped in wells containing one anti-CD137 Fab-Y (Fab-peptide) (10 μg/mL) to assess association of the antibody to each of the peptides (600 seconds), followed by dissociation in kinetics buffer (600 seconds). Anti-CD9 Fab-Y (7485) was used as negative control during the antibody association step. A new set of 8 biosensors was used to repeat this process for each of the anti-CD137 Fab-Y antibodies (8475, 8476, 8479, 11420, 11175, 11176, 11569, 11568, 11566).

TABLE 7

| | Amino acids | |
|---|---|---|
| Peptide | (with reference to SEQ ID NO: 1) | Domain in CD137 |
| P1 | 25-68 | CRD1 |
| P2 | 25-105 | CRD1-2 |
| P3 | 25-162 | CRD1-4 |
| P4 | 46-162 | CRD2-4 |
| P5 | 87-162 | CRD3-4 |
| P6 | 106-162 | CRD4 |

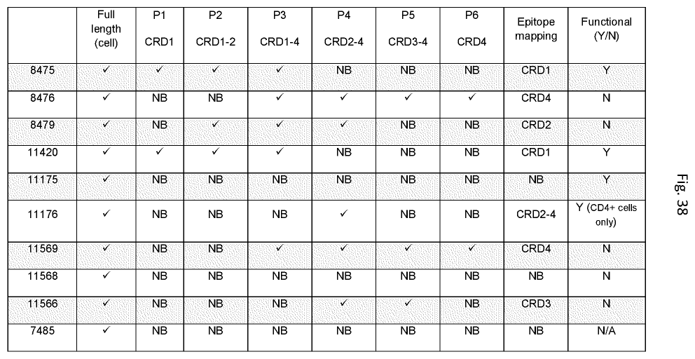

Binding data was acquired using the Octet® RED384 System (FortéBio). FIG. 38 shows the binding profile of Fab-Y antibodies to Fc-fusion CD137 peptides (P1-P6) and the corresponding functional response as a bispecific with CD9 as identified herein. Epitope mapping to each CRD was inferred from binding to the different peptides. A positive functional response is considered to be the capacity to increase CD25 expression on T cells by greater than 0.5 Log 2 fold change in 3 donors when added as anti-CD137 and anti-CD9 specificity.

As illustrated by FIG. 38, bispecific antibodies according to the present invention bind at least CRD1 of CD137 and are capable to generate a functional response when combined on the same molecule with antibodies binding CD9.

In particular, the bispecific antibody preferably binds within amino acids 25 to 68 of SEQ ID NO: 1, more preferably within amino acids 25 to 45 of SEQ ID NO: 1.

Antibody 11175 binds full length cell-expressed CD137 and is functional but does not bind any of the CRD domains (either independently expressed or as a combination). This antibody binds a natural conformational epitope that cannot be adequately represented by recombinant synthetic peptides.

The same analysis was performed to identify the regions of CD9 which are responsible for such function. CD9 contains two extracellular loops: a short extracellular loop (loop 1: 34-55 in SEQ ID NO:2) and a long extracellular loop (loop 2: 112-195 in SEQ ID NO:2). We used biolayer interferometry to assess binding of our panel of CD9 Fab-Y antibodies (selected for ability to bind full-length CD9 expressed on cells) to the long extracellular loop 2 peptide, using the Octet® RED384 System (FortéBio) with anti-hIgG Fc Capture (AHC) Biosensors (FortéBio) at room temperature. Firstly, an array of 8 sensors was dipped in kinetics buffer (PBS 0.1%, BSA 0.02%, Tween 20) for 120 seconds to provide a baseline signal. Sensors were then moved to wells containing 200 μl of recombinant human CD9 long extracellular loop 2-human Fc fusion protein (10015-CD, R&D Systems®) at 2 μg/mL in kinetics buffer to immobilize the protein to the biosensor (100 seconds), followed by a second baseline step (180 seconds) in kinetics buffer to equilibrate the biosensors now coated with the CD9 long extracellular loop 2-human Fc fusion protein. No detachment of the peptide was observed during this step. Sensors were then dipped in wells containing one of the anti-CD9 Fab (10 μg/mL) to evaluate association of each antibody to CD9-loop2 (120 seconds), followed by dissociation in kinetics buffer (600 seconds). Anti-CD137 Fab-Y (11175) was used as negative control during the antibody association step. A new set of 8 biosensors was used to repeat this process for each of the anti-CD9 antibodies (7270, 7271, 7272, 7485,7486, 7489, 7491).

As shown in Table 8, all the anti-CD9 antibodies tested bind to the long extracellular loop 2 of CD9 and all are functional when combined with an anti-CRD1 CD137 antibodies, such as the one used herein, anti-CD137 8475. A positive functional response is considered to be the capacity to increase CD25 expression on T cells by greater than 0.5 Log 2 fold change in 3 donors when added as a Fab-K_{D}-Fab with anti-CD137.

In particular, the bispecific antibodies according to the present invention bind within amino acids 25 to 68 of SEQ ID NO: 1, more preferably within amino acids 25 to 45 of SEQ ID NO: 1 and within amino acids 112 and 195 of SEQ ID NO: 2.

TABLE 8

| (+8475) | CD9 long extracellular loop 2 | Functional (Y/N) |
|---|---|---|
| 7270 | ✓ | Y |
| 7271 | ✓ | Y |
| 7272 | ✓ | Y |
| 7485 | ✓ | Y |
| 7486 | ✓ | Y |
| 7489 | ✓ | Y |
| 7491 | ✓ | Y |
| 11175 | NB | CD137 bivalent |

### Example 9: Comparison of the T Cell Proliferation Activation of CD137-CD9 Bispecific Antibodies Versus Ipilimumab and Nivolumab

Ipilimumab (an anti-CTLA-4 antibody) was the first checkpoint inhibitor to be approved in 2011 as a treatment for melanoma, closely followed by FDA approval of anti-PD1 directed antibodies, pembrolizumab and nivolumab in 2014 (Hargadon et al., International Immunopharmacol. 62:29-39 (2018)). Whilst there are still significant challenges in understanding differences in efficacy across patient groups, ranging from complete responses, to treatment relapse and even failure to respond, (Haslam and Prasad. JAMA Network Open.5:2e192535 (2019)), these molecules represent current clinically-validated references for immunotherapy in a range of cancer types and have been utilised in the present studies for benchmarking the activity of the novel bispecific antibodies described herein.

The effect of an anti-CD137-CD9 antibody on the level of CD71 and proliferation of CD4+ and CD8+ T cells was assessed in 4 PBMC donors and compared to equimolar quantities of Ipilimumab (Yervoy®) and Nivolumab (Opdivo®) as clinically tested reference molecules.

A grid of fusion proteins Fab-X and Fab-Y were created by diluting equimolar (1 μM) quantities of Fab-X (Fab-scFv) and Fab-Y (Fab-peptide) with specificity for CD9 and CD137 in TexMACS™ media (Miltenyi Biotec®) containing 100 U/mL penicillin/100 μg/mL streptomycin and 5% human AB Serum (Sigma-Aldrich). The Fab-X and Fab-Y fusion proteins were incubated together for 1 hour (in a 37° C., 5% CO₂ environment), at a final concentration of 500 nM. Negative control wells containing TexMACS™ media only were also generated alongside the Fab-X and Fab-Y wells. Ipilimumab and Nivolumab were diluted to 500 nM in TexMACS™ media containing 100 U/mL penicillin/100 μg/mL streptomycin and 5% human AB Serum (Sigma-Aldrich).

During this time, cryopreserved human PBMC isolated from platelet leukapheresis cones were thawed and washed in TexMACS™ media (containing 100 U/mL penicillin/100 μg/mL streptomycin and 5% human AB Serum (Sigma-Aldrich)) and resuspended in 10 mL PBS (ThermoFisher). 10 μl of 5 mM CellTrace™ Violet solution was added to the sample and mixed well by inversion. The cells were incubated at 37° C. for 20 minutes and washed by the addition of 45 mL PBS containing 10% heat inactivated foetal bovine serum (ThermoFisher Scientific). The cells were incubated for a further 5 minutes before centrifugation at 400×g for 5 minutes. The waste was removed, and the cells resuspended at 2.5×10⁶ cells/mL. The PBMC were then seeded into 96-well U-bottom tissue culture plates (Costar®) at 60 μl/well (1.5×10⁵ PBMC). A total of 20 μl of Fab-X/Fab-Y bispecific antibodies were transferred to the plates containing 60 μL PBMC. The PBMC were then either left unstimulated by the addition of 20 μl of media or stimulated with 20 μl of soluble anti-CD3 (clone UCHT-1) (50 ng/mL final concentration) with and without 200 ng/mL anti-CD28 (clone CD28.2). This resulted in a final assay concentration of antibodies of 100 nM. The plates were then returned to a 37° C., 5% CO₂ environment for 6 days.

After 6 days the plates were centrifuged at 500×g for 5 minutes at 4° C. Cell culture conditioned media was transferred from the cell pellets to fresh plates and frozen at ⁻80° C. Cells were washed with 60 μl FACS buffer by centrifugation, followed by resuspension of the pellets by shaking the plates at 1800 rpm for 30 seconds. The cells were stained with a cocktail of fluorescently labelled antibodies as listed in Table 9 and incubated at 4° C. in the dark for 30 minutes. After this time, cells were washed with FACS buffer and fixed with 2% paraformaldehyde for one hour at 4° C. before the addition of 150 μl FACS buffer. The plates were centrifuged at 500×g for 5 minutes, the fixation buffer aspirated to waste and the cells resuspended in a residual volume of 15 μl for acquisition on the iQue® Screener Plus (IntelliCyt®).

The data analysis software package ForeCyt™ (IntelliCyt®) was used to exclude CD14+ monocytes, followed by the identification of the CD4+ and CD8+ T cells. For each cell population the cellular expression of CD71 was measured as reported median fluorescent intensity values. The level of CellTrace™ Violet was also measured to identify cells having undergone cell division.

The data were then used to calculate the Log 2 fold changes in CD71 expression and the number of divided CD4 and CD8 positive T cells relative to the control wells.

TABLE 9

| Epitope | Fluorophore | Clone | Source | Dilution |
|---|---|---|---|---|
| CD14 | PE/Cy7 | M5E2 | BD Pharmagen ® | 80 |
| CD8 | BV605 | RPA-T8 | BioLegend ® | 40 |
| CD4 | FITC | RPA-T4 | BioLegend ® | 80 |
| CD71 | PE | M-A712 | BioLegend ® | 160 |
| Live/Dead | Near IR | | Thermo Scientific ® | 1000 |

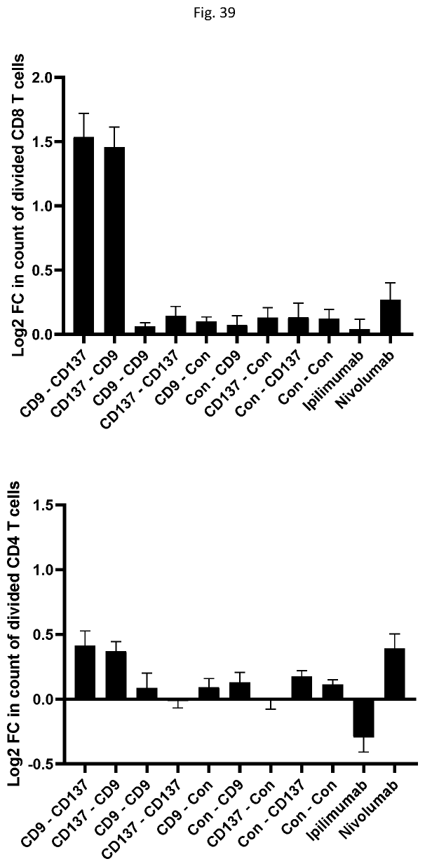

CD137-CD9 bispecific Fab-K_{D}-Fab antibodies led to increases in the number of dividing CD8+ T cells when PBMC cultures were treated in combination with 50 ng/mL anti-CD3 (UCHT-1) for 6 days. This increase was less apparent in the CD4⁺ T cells of these cultures, however small increases could be detected (FIG. 39). Bivalent and monovalent controls did not lead to any increase in the number of dividing CD4⁺ or CD8⁺ T cells. Ipilimumab did not have any impact of the number of dividing T cells, while Nivolumab did lead to small increases, but to a much smaller degree than that observed for the bispecific antibody on CD8⁺ T cells.

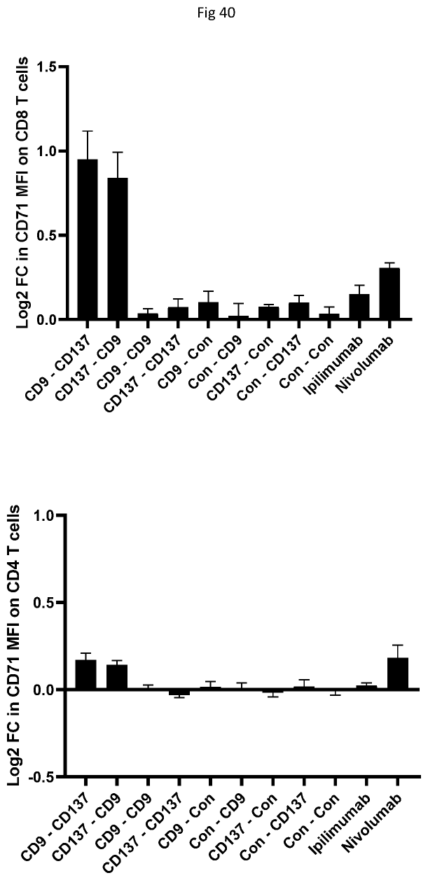

The Log 2 fold change in the CD71 MFI of the activation marker on CD8⁺ and CD4⁺ T cells showed a very similar result (FIG. 40), supporting the finding that the CD137-CD9 bispecific antibody increased the activation of CD8⁺ T cells, in particular when in combination with a sub-maximal anti-CD3 stimulus, while the bivalent and monovalent controls did not.

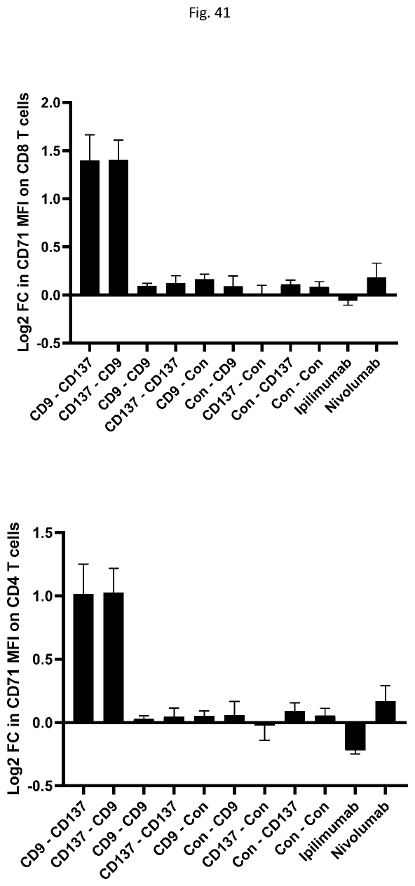

These constructs were also tested in combination with a sub-maximal anti-CD3 stimulus in combination with of anti-CD28 co-stimulation (clone 28.2). Increases in CD71 MFI on both CD8⁺ and CD4⁺ T cells could be detected (FIG. 41), while no increase was measured in response to the bivalent and monovalent controls or commercial comparators.

### Example 10: Comparison of Fab-X/Fab-Y Bispecific Antibodies with Therapeutic Formats

As already shown in Example 3, the Fab-X/Fab-Y bispecific antibodies against CD137 and CD9 achieve the same effect when converted into a therapeutic format such as an IgG format. To investigate if such effect is restricted to an IgG format, the variable regions of the Fab-X and Fab-Y fusion proteins were cloned into a BYbe™ format using established methods available in the public domain. Bivalent Control BYbe™ was also generated. The functionality of targeting CD9 and CD137 in the different bispecific formats was tested by comparing them in an anti-CD3 (clone UCHT-1) stimulated PBMC assay.

Fab-X/Fab-Y bispecific antibodies were created by adding equimolar quantities of Fab-X (Fab-scFv) and Fab-Y (Fab-peptide) with specificity for CD9 and CD137 in TexMACS™ media (Miltenyi Biotec®) containing 100 U/mL penicillin/100 μg/mL streptomycin. The Fab-X and Fab-Y fusion proteins were incubated together for 1 hour (in a 37° C., 5% CO₂ environment), at a final concentration of 500 nM. BYbe™ constructs were diluted to 500 nM in TexMACS™ media containing 100 U/mL penicillin/100 μg/mL streptomycin. Negative control wells containing TexMACS™ media only were also generated alongside.

During the incubation, cryopreserved human PBMC isolated from platelet leukapheresis cones were thawed and washed in TexMACS™ media and resuspended at 2.5×10⁶ cells/mL. The PBMC were then seeded into 96-well U-bottom tissue culture plates (Costar®) at 60 μl/well (1.5×10⁵ PBMC). A total of 20 μl of Fab-X/Fab-Y bispecific antibodies or BYbe™ constructs were transferred to the plates containing 60 μL PBMC. The PBMC were then stimulated with 20 μl of soluble anti-CD3 (clone UCHT-1) at 10 ng/mL final concentration. This resulted in a final assay concentration of antibodies of 100 nM. Negative control wells had 20 μl of TexMACS™ media added instead of antibody. The plates were then returned to a 37° C., 5% CO₂ environment for 48 hours.

After 48 hours the plates were washed with 100 μl FACS buffer and centrifuged at 500×g for 5 minutes at 4° C. The cells were washed with 180 μl FACS buffer followed by centrifugation and by resuspension of the pellets by shaking the plates at 1800 rpm for 30 seconds. The cells were stained with a cocktail of fluorescently labelled antibodies as listed in Table 10 and incubated at 4° C. in the dark for 1 hour. After this time, cells were washed with FACS buffer, and fixed with 2% paraformaldehyde for one hour at 4° C. before the addition of 150 μl FACS buffer. The plates were centrifuged at 500×g for 5 minutes, the fixation buffer aspirated to waste and the cells resuspended in a residual volume of 15 μl for acquisition on the iQue® Screener Plus (IntelliCyt®).

TABLE 10

| Epitope | Fluorophore | Clone | Source | Dilution |
|---|---|---|---|---|
| CD56 | APC | HCD56 | BioLegend ® | 40 |
| CD14 | PE/Cy7 | M5E2 | BD Pharmagen ® | 80 |
| CD8 | BV650 | RPA-T8 | BioLegend ® | 80 |
| CD4 | BV605 | RPA-T4 | BD Horizon ® | 40 |
| CD19 | PE-Cy5 | HIB19 | BD Pharmagen ® | 20 |
| CD45RO | BV786 | UCHL1 | BD Horizon ® | 40 |
| CD71 | FITC | M-A712 | BD Pharmagen ® | 20 |
| CD25 | BV510 | M-A251 | BD Horizon ® | 40 |
| CD137 | PE | 4B4-1 | BD Pharmagen ® | 20 |
| Live/Dead | Near IR | | Thermo Scientific ® | 1000 |

The data analysis software package ForeCyt™ (IntelliCyt®) was used to gate on CD14⁺ monocytes, CD56⁺ NK cells, CD19⁺ B cells, CD4⁺ and CD8⁺ memory and naïve T cells. For each cell population the cellular expression of CD25 was measured as reported median fluorescent intensity values. The data were then used to calculate the Log 2 fold changes of expression relative to control well values.

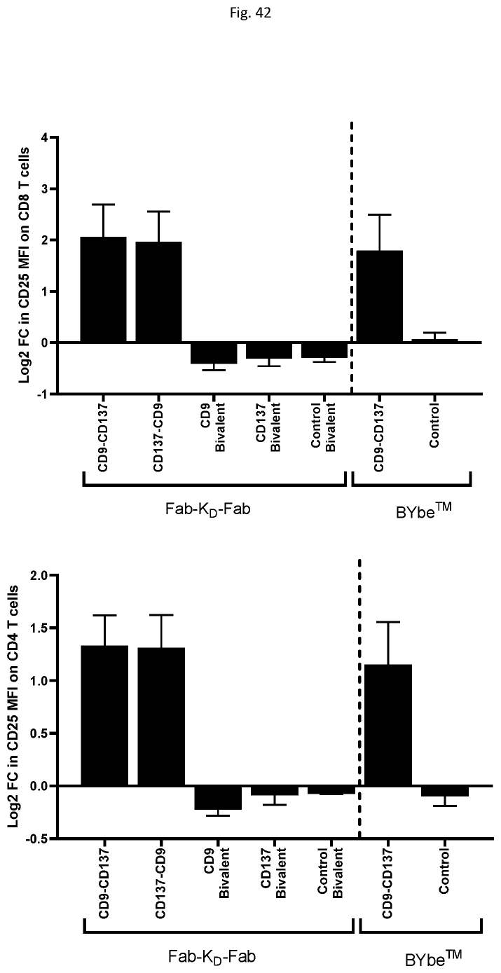

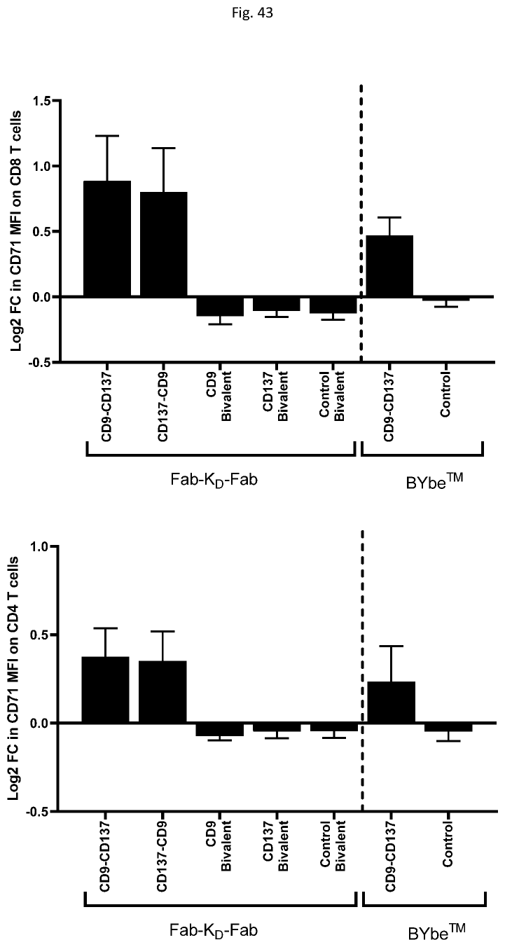

As in previous examples CD137-CD9 bispecific antibodies, as Fab-K_{D}-Fabs, led to increases in the level of activation markers CD25 (FIG. 42) and CD71 (FIG. 43) of CD8⁺ and CD4⁺ T cells when treated in combination with a sub-maximal anti-CD3 stimulus over 48 hours. The bivalent controls (two antigen-binding portions either binding only CD9 or only CD137) did not cause any increase in activation marker expression. When tested alongside the bispecific antibodies Fab-X/Fab-Y, the BYbe™ antibodies led to comparable increases in activation marker expression on both CD8⁺ and CD4⁺ T cells, demonstrating that the functional effect can be replicated in an alternative antibody formats such as IgG and formats comprising covalently linked antibody fragments.

### Example 11: Evaluation of the Effect of CD137-CD9 Bispecific on Peptide Stimulated Melanoma Patient PBMC

Peripheral blood mononuclear cells (PBMC) from patients with melanoma were isolated on Ficoll-Paque® PLUS by density centrifugation. PBMC were cultured at 2×10⁵ cells/well and stimulated with a peptide pool comprising sequences from known melanoma tumour-associated antigens (PepMix™ collection Melanoma PM-CMel, JPT Innovative Peptide Solutions). Cells were cultured in the absence (1% PBS vehicle control), or presence of test antibodies. Purified Fab-X and Fab-Y were incubated together for 60 minutes (in a 37° C./5% CO₂ environment) at an equimolar concentration. The final concentration of both Fab-K_{D}-Fab complex and IgG molecules was 100 nM. Where sufficient cells were available, a melanoma peptide-stimulated vehicle control (1% PBS) condition was also included. Cells were cultured in RPMI 1640 medium (with sodium bicarbonate and L-glutamine, Sigma-Aldrich) with 100 U/mL penicillin/100 μg/mL streptomycin (Sigma-Aldrich) and 5% (v/v) heat-inactivated human AB serum (Sigma-Aldrich) in 96-well round bottom plates (Sarstedt) for 6 days at 37° C./5% CO₂ in 95% air.

Following 6 days in culture, plates were centrifuged, and conditioned medium removed and stored at −20° C. prior to cytokine analysis. The remaining cells were stained with anti-human CD3-BV510, CD4-PeCy7, CD8-FITC, CD56-BV421, CD25-APC and CD71-PE and Zombie NIR™ fixable viability dye (BioLegend), and then fixed using FoxP3 staining buffer set (BD Biosciences). Following fixation, cells were washed and resuspended in buffer (PBS plus 5% foetal bovine serum). Results were analysed using FlowJo™ version 10. Absolute concentrations of IFN-γ, TNFα, IL-2 and IL-10 in the conditioned medium were measured by Luminex® (Biotechne/R&D Systems) according to manufacturer's instructions. Samples were diluted 1:2 and analysed using a Bio-Plex® 200 reader and Bio-Plex® Manager™ software.

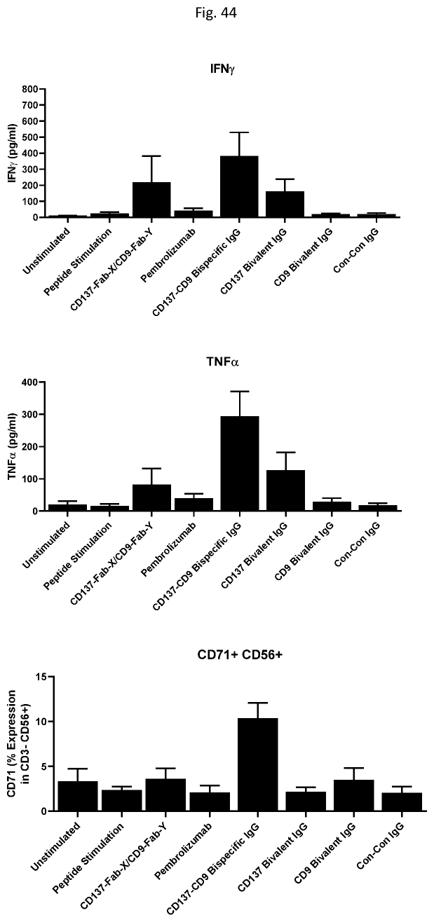

FIG. 44 summarises the effects of all antibodies and treatments on the release of IFNγ, TNFα and NK cell activation from peptide-stimulated melanoma PBMC from patients. The melanoma antigen peptide mix failed to stimulate increased levels of IFNγ, TNFα or effect NK cell activation in these samples, reflecting an immunosuppressed phenotype characteristic of previous observations in melanoma. However, both the CD137-CD9 Fab-K_{D}-Fab complex and CD137-CD9 IgG were able to stimulate elevated release of both IFNγ and TNFα in all donors tested. The CD137 bivalent showed similar effects to the CD137-CD9 Fab-K_{D}-Fab with respect to the effect on IFNγ and TNFα release, but this was of lower magnitude than the effect of the CD137-CD9 bispecific IgG. With respect to elevated CD71 expression as a marker of NK cell activation, the CD137-CD9 bispecific IgG, was the only treatment that was capable of enhancing NK cell activation. Under these experimental conditions, pembrolizumab (an anti-PD1 inhibitory antibody) which blocks the PD-1 mediated down-regulation of T cell and myeloid cell activation in the tumour microenvironment, did not exhibit any stimulatory activity with respect to either cytokine release or NK cell activation, clearly evidencing the superior and advantageous effect of a bispecific antibody against CD137 and CD9.

### Example 12: Evaluation of the Effect of CD137-CD9 Bispecific in a Colorectal Tumour Micro-Environment Model

The BioMAP® Colorectal Cancer (CRC) panel (Eurofins) models the interactions between the immune-stromal (fibroblasts) and immune-vascular (endothelial cells) environments in the context of colorectal cancer (HT-29 colorectal adenocarcinoma cell line). The interactions between tumour cells, stimulated peripheral blood mononuclear cells (PBMC), and the host stromal network are modelled in the StroHT29 system, whilst the VascHT29 system captures the interactions between tumour cells, activated PBMC and vascular tissue. The biomarkers selected for the BioMAP® CRC panel reflect a range of activities related to inflammation, immune-function, tissue remodelling and metastasis, modelling tumour-mediated immune suppression that occurs in the tumour microenvironment (TME) of cancer patients. The StroHT29 system is comprised of the HT-29 colorectal adenocarcinoma cell line, human neonatal dermal fibroblasts (HDFn) and PBMC. The VascHT29 system is comprised of the HT-29 colorectal adenocarcinoma cell line, human umbilical vein endothelial cells (HUVEC) and PBMC. Both systems are stimulated by sub-mitogenic levels of SEB and the stimulation conditions are optimised to activate or prime T cells, but not drive T cell proliferation.

Human primary cells used in the BioMAP® systems were at early passage (passage 4 or earlier) to minimise adaptation to cell culture conditions and preserve physiological signalling responses. Primary cells were commercially purchased, pooled from multiple donors (n=3-6), and handled according to manufacturer's instructions. The HT-29 colorectal adenocarcinoma cell line was purchased from American Type Culture Collection (ATCC).

Adherent cell types were cultured in 96-well plates until confluent, followed by the addition of PBMC. Test antibodies were prepared in PBS and added at 100 nM, 1 hour prior to stimulation for 48 hours. Each plate also contained several controls, including negative unstimulated controls vehicle controls and drug reference controls.

Direct ELISA was used to measure biomarker levels of cell-associated and cell membrane targets. Soluble factors from conditioned medias are quantified using either HTRF® detection, multiplex electrochemiluminescence assay or capture ELISA. Effects of test agents on cell viability (cytotoxicity) were measured by sulforhodamine B (SRB) for adherent cells (48 hours), and by AlamarBlue® reduction for PBMC (42 hours). All test agents were tested at 100 nM in triplicate. Data acceptance criteria were based on plate performance (% CV of controls <10%) and the performance of controls across assays with a comparison to historical controls.

Biomarker measurements were profiled in triplicate for antibody-treated samples, and then averaged and divided by the average of vehicle control samples (at least 6 vehicle controls from the same plate) to generate a fold change that is then Log 2 transformed. Statistical p values were calculated from unpaired t-test statistics of raw data values compared to vehicle controls. Significant changes in biomarkers are defined as changes induced by the test or reference molecules, that have an effect size >20% compared to the vehicle control (Log 2 ratio >0.2) and a p value <0.01.

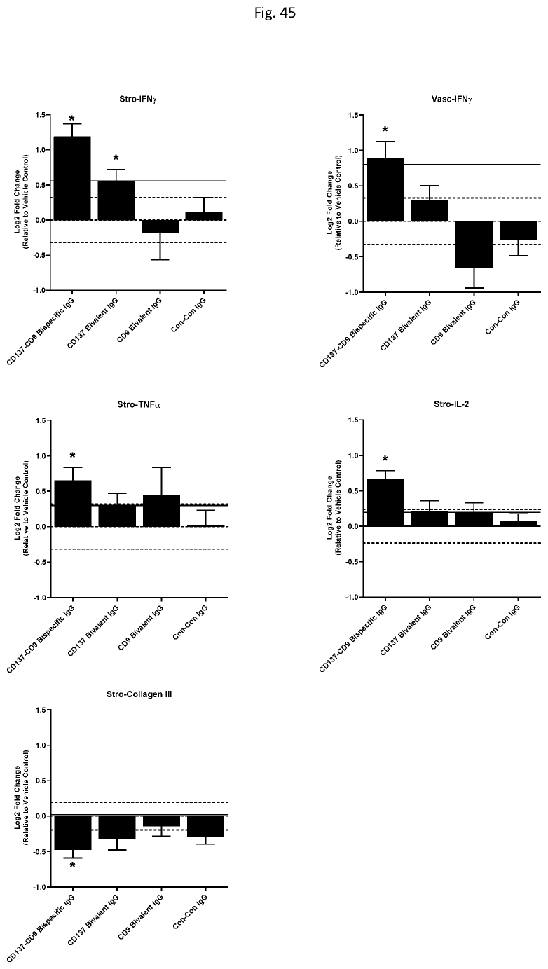

CD137-CD9 bispecific IgG (generated according to Example 3) was not cytotoxic to any cell type in either the StroHT29 or VascHT29 systems. CD137-CD9 bispecific IgG was associated with increased inflammation and immune-related activities, including increases in IFNγ, IL-2 and TNFα in the StroHT29 system indicating an immune restorative capacity consistent with the profile of approved anti-PD-1 antibodies, such as pembrolizumab which was used as a reference molecule in this assay (FIG. 45). Additionally, CD137-CD9 bispecific IgG also decreased collagen III, possibly indicating a potential matrix-inhibitory/anti-fibrotic effect that could allow increased immune infiltration into the TME. The CD137 bivalent IgG (similar to urelumab anti-CD137 IgG), inhibited IFNγ production in the StroHT29 system, but this effect was approximately 50% of that observed with the CD137-CD9 bispecific molecule, suggesting a greatly enhanced bispecific-mediated effect. Conversely, the CD9 bivalent IgG showed no indication of immune restoration via induction of pro-inflammatory cytokines such as IFNγ in the StroHT29 system, but interestingly did inhibit IFNγ production in the VascHT29 system.

1. An antibody which comprises a first antigen-binding portion binding CD137 and a second antigen-binding portion binding CD9.

2. The antibody according to claim 1, wherein each of the antigen-binding portions is a monoclonal antigen-binding portion.

3. The antibody according to claim 1, wherein each of the antigen-binding portions is independently selected from a Fab, a Fab′, a scFv or a VHH.

4. The antibody according to claim 1, wherein the antigen-binding portions are the antigen-binding portions of an IgG.

5. The antibody according to claim 1, wherein the antibody is chimeric, human or humanised.

6. The antibody according to claim 1, wherein the antibody comprises a heavy chain constant region selected from an IgG1, an IgG2, IgG3 or an IgG4 isotype, or a variant thereof.

7. The antibody according to claim 1, wherein the antibody further comprises at least an additional antigen-binding portion.

8. The antibody according to claim 7, wherein the additional antigen-binding portion is capable of increasing the half-life of the antibody.

9. The antibody according to claim 8, wherein the additional antigen-binding portion binds albumin.

10. The antibody according to claim 1, wherein the first antigen-binding portion binds CD137 in CD137 domain CRD1.

11. The antibody according to claim 1, wherein the second antigen binding portion binds CD9 in CD9 loop 2.

12. The antibody according to claim 1, wherein the first antigen-binding portion binding CD137 comprises a first heavy chain variable region and a first light chain variable region, wherein the second antigen-binding portion binding CD9 comprises a second heavy chain variable region and a second light chain variable region, and wherein:
a. The first heavy chain variable region comprises a CDR-H1 comprising SEQ ID NO: 3, a CDR-H2 comprising SEQ ID NO: 4 and a CDR-H3 comprising SEQ ID NO: 5; and
b. The first light chain variable region comprises a CDR-L1 comprising SEQ ID NO: 6, a CDR-L2 comprising SEQ ID NO: 7 and a CDR-L3 comprising SEQ ID NO: 8; and
c. The second heavy chain variable region comprises a CDR-H1 comprising SEQ ID NO: 9, a CDR-H2 comprising SEQ ID NO: 10 and a CDR-H3 comprising SEQ ID NO: 11; and
d. The second light chain variable region comprises a CDR-L1 comprising SEQ ID NO: 12, a CDR-L2 comprising SEQ ID NO: 13 and a CDR-L3 comprising SEQ ID NO: 14;
or
e. The first heavy chain variable region comprises SEQ ID NO: 15 and the first light chain variable region comprises SEQ ID NO: 17; and the second heavy chain variable region comprises SEQ ID NO: 19 and second light chain variable region comprises SEQ ID NO: 21;
or
f. The first heavy chain variable region is encoded by a nucleotide sequence comprising SEQ ID NO: 16 and the first light chain variable region is encoded by a nucleotide sequence comprising SEQ ID NO: 18; and the second heavy chain variable region is encoded by a nucleotide sequence comprising SEQ ID NO: 20 and second light chain variable region is encoded by a nucleotide sequence comprising SEQ ID NO: 22.

13. A pharmaceutical composition comprising the antibody according to claim 1 and one or more pharmaceutically acceptable excipients.

14. (canceled)

15. (canceled)

16. (canceled)

17. A method for treating a subject afflicted with cancer, an infectious disease, or a combination thereof, comprising administering to the subject a pharmaceutically effective amount of the antibody according to claim 1 or a pharmaceutical composition comprising the antibody and one or more pharmaceutically acceptable excipients.

18. The method according to claim 17, wherein the antibody or the composition is administered concomitantly or sequentially to one or more additional cancer therapies.

19. The method according to claim 17, wherein the subject is afflicted with cancer.

20. The method according to claim 19, wherein the antibody or the pharmaceutical composition is administered concomitantly or sequentially to one or more additional cancer therapies.


SEQUENCE LISTING (22 sequences)

SEQ ID NO: 1 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 2 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 3 - protein, 10 aa, Artificial Sequence
  CDR-H1 VR8475
GFSLSTYAMS

SEQ ID NO: 4 - protein, 16 aa, Artificial Sequence
  CDR-H2 VR8475
AIWSGGTTDYTSWAKG

SEQ ID NO: 5 - protein, 12 aa, Artificial Sequence
  CDR-H3 VR8475
MAVIFNAYTFDS

SEQ ID NO: 6 - protein, 11 aa, Artificial Sequence
  CDR-L1 VR8475
QASQNIYSYLA

SEQ ID NO: 7 - protein, 7 aa, Artificial Sequence
  CDR-L2 VR8475
GASTLAS

SEQ ID NO: 8 - protein, 13 aa, Artificial Sequence
  CDR-L3 VR8475
QQGHSSGNVGNNV

SEQ ID NO: 9 - protein, 10 aa, Artificial Sequence
  CDR-H1 VR7272
GFSLSSYAMG

SEQ ID NO: 10 - protein, 16 aa, Artificial Sequence
  CDR-H2 VR7272
AIGSITATGYARWAKG

SEQ ID NO: 11 - protein, 12 aa, Artificial Sequence
  CDR-H3 VR7272
EIYVGSAYAFDI

SEQ ID NO: 12 - protein, 11 aa, Artificial Sequence
  CDR-L1 VR7272
QASQSISNYLA

SEQ ID NO: 13 - protein, 7 aa, Artificial Sequence
  CDR-L2 VR7272
LASTLAS

SEQ ID NO: 14 - protein, 11 aa, Artificial Sequence
  CDR-L3 VR7272
QQGYIDNVNKG

SEQ ID NO: 15 - protein, 117 aa, Artificial Sequence
  VH VR8475
QSVEESGGRLVTPGTPLTLTCTVSGFSLSTYAMSWVRQAPGKGLEWIGAIWSGGTTDYTSWAKGRFTISKASTTVDLKITSPTTEDTATYFCVRMAVIFNAYTFDSWGPGTLVTVSS

SEQ ID NO: 16 - DNA, 424 bp, Artificial Sequence
  VH nucl VR8475
aagcttcgaagccaccatggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtacgtatgcaatgagctgggtccgccaggctccagggaaggggctggagtggatcggagcgatttggagtggtggtaccacggactacacgagctgggcgaaaggccgattcaccatctccaaagcctcgaccacggtggatctgaaaatcaccagtccgacaaccgaggacacggccacctatttctgtgtcagaatggcagtgatttttaatgcttacacttttgattcctggggcccaggcaccctggtcaccgtctcgagt

SEQ ID NO: 17 - protein, 111 aa, Artificial Sequence
  VL VR8475
AYDMTQTPSSVSAAVGGTVTIKCQASQNIYSYLAWYQQKPGQRPKLLIYGASTLASGVPSRFKGSGSGTDFTLTISDLECDDAATYYCQQGHSSGNVGNNVFGGGTEVVVK

SEQ ID NO: 18 - DNA, 421 bp, Artificial Sequence
  VL nucl VR8475
aagcttcgaagccaccatgaacatgagggcccccactcagctgctggggctcctgctgctctggctcccaggtgccagatgtgcctatgatatgacccagactccatcctccgtgtctgcagctgtgggaggcacagtcaccatcaagtgccaggccagtcagaacatttacagttacttagcctggtatcagcagaaaccagggcagcgtcccaagctcctgatctatggtgcgtccactctggcatctggggtcccatcgcggttcaaaggcagtggatctgggacagatttcactctcaccatcagcgacctggagtgtgacgatgctgccacttactactgtcaacagggccatagtagtggtaatgttggaaataatgttttcggcggagggaccgaggtggtggtcaaacgtacg

SEQ ID NO: 19 - protein, 117 aa, Artificial Sequence
  VH VR7272
QSLEESGGRLVTPGTPLTLTCTVSGFSLSSYAMGWVRQAPGKGLEWIGAIGSITATGYARWAKGRFSISKTSTTVDLKMTSPTTEDTATYFCAREIYVGSAYAFDIWGPGTLVTVSS

SEQ ID NO: 20 - DNA, 424 bp, Artificial Sequence
  VH nucl VR7272
aagcttcgaagccaccatggagactgggctgcgctggcttctcctggtcgctgtgctcaaaggtgtccagtgtcagtcgctggaggagtccgggggtcgcctggtcacgcctgggacacccctgacactcacctgcacagtctctggattctccctcagtagctatgcaatgggctgggtccgccaggctccagggaaggggctggagtggatcggagccattggtagtattactgccactggctacgcgcgctgggcaaaaggccgattcagcatctccaagacctcgaccacggtggatctgaaaatgaccagtccgacaaccgaggacacggccacctatttctgtgccagagagatttatgttgggtctgcttatgcctttgacatctggggcccaggcaccctggtcaccgtctcgagt

SEQ ID NO: 21 - protein, 109 aa, Artificial Sequence
  VL VR7272
AYDMTQTPASVEVAVGDTVTIKCQASQSISNYLAWYQQKPGQPPKLLIYLASTLASGVPSRFKGSGSGTEFTLTISDLECADAATYYCQQGYIDNVNKGFGGGTEVVVK

SEQ ID NO: 22 - DNA, 415 bp, Artificial Sequence
  VL nucl VR7272
aagcttcgaagccaccatggacacgagggcccccactcagctgctggggctcctgctgctctggctcccaggtgccagatgtgcctatgatatgacccagactccagcctctgtggaggtagctgtgggagacactgtcaccatcaagtgtcaggccagtcagagcattagtaactacttagcctggtatcagcagaaaccagggcagcctcccaagctcctgatctatctggcatctactctggcatctggggtcccatcgcggttcaaaggcagtggatctgggacagagttcactctcaccatcagcgacctggagtgtgccgatgctgccacttactattgtcaacagggttatattgataatgttaataaaggtttcggcggagggaccgaggtggtggtcaaacgtacg